(19) United States
(12) Patent Application Publication
(10) Pub. No.: US 2025/0019780 A1
(43) Pub. Date: Jan. 16, 2025

(54) METHOD FOR DETERMINING DNA CONCENTRATION IN DNA VIRUS

(71) Applicant: Access Medical Systems, Ltd., Palo Alto, CA (US)

(72) Inventors: Robert F. Zuk, Menlo Park, CA (US); Samuel Yang, San Jose, CA (US); Alex Ho Fai Lee, Fremont, CA (US); Indrani Chakraborty, Fremont, CA (US); Pu Li, Los Altos, CA (US); Wai Choi, San Mateo, CA (US)

(21) Appl. No.: 18/900,261

(22) Filed: Sep. 27, 2024

Related U.S. Application Data
(63) Continuation-in-part of application No. PCT/US2023/065854, filed on Apr. 18, 2023.
(60) Provisional application No. 63/363,145, filed on Apr. 18, 2022.
(60) Provisional application No. 63/363,972, filed on May 2, 2022.
(60) Provisional application No. 63/365,313, filed on May 25, 2022.
(60) Provisional application No. 63/378,816, filed on Oct. 7, 2022.
(60) Provisional application No. 63/586,885, filed on Sep. 29, 2023.

(51) Int. Cl. C12Q 1/70; G01N 33/53; G01N 33/569; G01N 33/58

(52) U.S. Cl. CPC C12Q 1/701; G01N 33/5308; G01N 33/56983; G01N 33/582; G01N 2333/015

(57) ABSTRACT

The present invention is directed to a method of determining DNA concentration in a DNA virus. The invention features three basic steps. The initial step is the specific capture of a defined amount of virus capsid particles on a first solid phase. The second step is lysis of the capsid to release the virus DNA from the first solid phase into a lysis solution. After separating the lysis solution from the first solid phase, the third step is contacting the lysis solution with a second solid phase. The second solid phase captures total DNA derived from the captured capsid. The present invention is also directed to a method for measuring the percentage of full virus capsid, comprising first determining the ssDNA concentration in viruses, and then converting the ssDNA concentration to percentage of full virus capsid using a calibration curve having DNA concentration plotted against standards of % of full capsids.

### CROSS-REFERENCE TO RELATED APPLICATION(S)

This application is a continuation-in-parts of PCT/US2023/065854, filed Apr. 18, 2023; which claim priority to U.S. Provisional Application Nos. 63/363,145, filed Apr. 18, 2022; 63/363,972, filed May 2, 2022; 63/365,313, filed May 25, 2022, and 63/378,816, filed Oct. 7, 2022. This application also claims priority to U.S. Provisional Application No. 63/586,885, filed Sep. 29, 2023. The above identified applications are incorporated herein by reference in their entireties.

### FIELD OF THE INVENTION

The present invention provides methods to determine DNA concentration in DNA viruses such as adeno-associated viruses (AAVs), and methods to determine empty verses full virus capsids.

### BACKGROUND OF THE INVENTION

Adenoviruses are medium-sized, nonenveloped viruses with an icosahedral nucleocapsid containing a double-stranded DNA genome. The adenovirus particle consists of an icosahedral protein shell (capsids) surrounding a protein core that contains the linear, double-stranded DNA genome. The shell, which is 70 to 100 nm in diameter, is made up of 252 structural capsomeres.

Herpes simplex virus type 1 (HSV-1) has a large linear double-stranded DNA genome in an icosahedral capsid shell, a cell-derived lipid envelope and a proteinaceous tegument layer. There are over fifty viral proteins and many host proteins identified in HSV-1 virions.

Adeno-associated viruses (AAVs) have emerged as vectors of choice for gene therapy clinical trials because of their long-term expression and lack of pathogenicity in humans.1. The wild-type adeno-associated virus (AAV) consists of a single-stranded DNA genome up to 4.8 kb in size that is flanked by inverted terminal repeats (ITRs), and this genome is encapsidated within a protein shell that is assembled from 60 proteins at a molar VP1:VP2:VP3 ratio of approximately 1:1:10.

AAV vectors that contain a DNA transgene packaged into a protein capsid have shown tremendous therapeutic potential in recent years. An inherent characteristic of the AAV manufacturing process is production of capsids that are not packaged with the therapeutic transgene and are therefore referred to as empty capsids. In general, up to 95% of the capsids produced upstream in the cell culture may be empty capsids, and this percentage has been shown to vary significantly between independent vector preparations. In its natural life cycle, wild-type AAVs have also been shown to produce a large proportion of empty capsids. The clinical effect of these empty capsids is not well understood, but it has been suggested that there could be elevated immune responses to high concentrations of viral particles and potential impairment of potency through receptor competition. Empty capsids are unable to provide the intended therapeutic benefit and are therefore considered to be a product-related impurity. In addition to empty capsids, a heterogeneous population of partially filled (or intermediate) capsids may also be produced during the manufacturing process, containing packaged process-related impurities or truncated genomes. Therefore, it is essential to have analytical techniques that are capable of providing information regarding the content distribution of AAV capsids, referred to capsid content.

Effectively quantifying AAV titers and determining the DNA transgene content by empty/full (E/F) capsid ratios is a major challenge for developing and manufacturing gene therapy vectors. A list of currently available tools includes AUC, TEM, ELISA, PCR-based methods and more. TEM (transmission electron microscopy) and AUC (analytical ultra-centrifugation) methods are based the capsid size difference under the assumption there is a correlation to the DNA transgene content. qPCR is an established technique but can take hours and require development and optimization for each AAV vector. Digital droplet PCR (ddPCR) is more precise than qPCR but has a smaller dynamic range and requires exact sample dilutions.

Current methods require that the AAV sample be purified prior to analysis. AAV purification methods are multi-step, labor intensive, and time consuming. A typical AAV purification process includes multiple steps involving filtration, column chromatography, and centrifugation. For example, a commercial scale AAV production includes the steps: thaw HEK cells, expand HEK cells and transfect, mechanical cell lysis (release vector from cells), freeze, depth filtration (clarification), cross flow filtration (concentration), lodixanol centrifugation (full capsid separation), affinity chromatography, and fill and finish. The complexity of the purification process is a major limitation for the accuracy, reproducibility, and adoption of E/F capsid measurements. Also, purified samples are not necessarily very pure, possibly containing trace contaminants capable of interfering with the analytical method.

There is a need to have analytical techniques that provides information regarding the content distribution of virus capsids, which is referred to as capsid contents.

### BRIEF DESCRIPTION OF THE DRAWINGS

FIG. 1A illustrates one example of a biosensor interferometer system suitable for the present method. FIGS. 1B and 1C each illustrates a biosensor interferometer probe.

FIGS. 2A-2B show first and second reflected light signals form a spectral interference pattern.

FIG. 3 shows a flow chart of preparing crosslinked-FICOLL® (copolymers of sucrose and epichlorohydrin) 400-SPDP.

FIG. 4 shows a flow chart of preparing streptavidin/HRP-Crosslinked FICOLL® conjugate.

FIG. 5 shows a flow chart of preparing anti-fluorescein-crosslinked FICOLL®.

FIG. 6 illustrates the steps in performing an assay for ssDNA.

FIG. 7 shows protocols in performing the assay for ssDNA illustrated in FIG. 6.

FIG. 8 presents a standard curve of M13 plasmid DNA. The wavelength shifts correspond to the bound HRP enzyme activity.

FIG. 9 shows the wavelength shift (nm) vs. time (second) of 24 replicates in BLI assays.

FIG. 10 outlines the 3 major steps of the ssDNA assay: AAV capture, lysis, then total AAV DNA measurement.

FIG. 11 shows typical nm shifts for capturing AAV at 4×1011 viral particles/mi, which approached plateau binding on a AAVX sensor after 30 minutes.

FIG. 12 depicts results of DNA concentration vs. % of full AAV capsids.

FIG. 13 illustrates the steps in performing a BLI assay for ssDNA with anti-DNA-HRP conjugate.

FIG. 14 shows the protocols in performing the BLI assay for ssDNA illustrated in FIG. 13.

FIG. 15 presents the M13 plasmid DNA dose response with the protocol of FIG. 14.

FIG. 16 shows wavelength shift (nm) vs. time (sec) of AAV capture at 3 different AAV concentrations.

FIG. 17 shows the ss DNA assay results (wavelength shift vs. time) of the captured AAV capsids of FIG. 16.

FIG. 18 shows a AAV standard curve prepared by known standards.

FIG. 19 shows ssDNA assay results of two AAV samples by magnetic particle capture and BLI detection.

FIG. 20 illustrates a flow chart in performing a fluorescent assay for ssDNA with biotin-anti-DNA and streptavidin-crosslinked-FICOLL®-Cys (SA-CxFICOLL-Cys). RFU: relative fluorescent unit.

FIG. 21 shows the protocols in performing a fluorescent assay for detecting ssDNA as illustrated in FIG. 21. Q buffer is PBS with 0.2% BSA, 0.02% Tween and 0.05% azide.

FIG. 22 illustrates a flow chart in performing a fluorescent assay for detecting ssDNA in an AAV sample with biotin-anti-DNA and streptavidin-crosslinked-FICOLL®-Cys (SA-CxFICOLL-Cys). RFU: relative fluorescent unit.

FIG. 23 shows the protocols in performing a fluorescent assay for detecting ssDNA in an AAV sample as illustrated in FIG. 22.

FIG. 24 shows a standard curve by plotting average RFU vs. % of full AAV in a sample.

### DETAILED DESCRIPTION OF THE INVENTION

### Definitions

Terms used in the claims and specification are to be construed in accordance with their usual meaning as understood by one skilled in the art except and as defined as set forth below.

“About,” as used herein, refers to within ±10% of the recited value.

An “analyte-binding” molecule, as used herein, refers to any molecule capable of participating in a specific binding reaction with an analyte molecule. Examples include but are not limited to, (i) antigen molecules, for use in detecting the presence of antibodies specific against that antigen; (ii) antibody molecules, for use in detecting the presence of antigens; (iii) protein molecules, for use in detecting the presence of a binding partner for that protein; (iv) ligands, for use in detecting the presence of a binding partner; or (v) single stranded nucleic acid molecules, for detecting the presence of nucleic acid binding molecules.

An “aspect ratio” of a shape refers to the ratio of its longer dimension to its shorter dimension.

A “binding molecule,” refers to a molecule that is capable to bind another molecule of interest.

“A binding pair,” as used herein, refers to two molecules that are attracted to each other and specifically bind to each other. Examples of binding pairs include, but not limited to, an antigen and an antibody against the antigen, a ligand and its receptor, complementary strands of nucleic acids, biotin and avidin, biotin and streptavidin, lectin and carbohydrates. Preferred binding pairs are biotin and streptavidin, biotin and avidin, fluorescein and anti-fluorescein, digioxigenin/anti-digioxigenin.

A “full AAV capsid” contains complete genomic materials, while an “empty AAV capsid” contains no genomic material.

“Immobilized,” as used herein, refers to reagents being fixed to a solid surface. When a reagent is immobilized to a solid surface, it is either be non-covalently bound or covalently bound to the surface.

“A monolithic substrate,” as used herein, refers to a single piece of a solid material such as glass, quartz, or plastic that has one refractive index.

A BLI “probe,” as used herein, refers to a monolithic substrate having as aspect ratio (length-to-width) of at least 2 to 1 with a thin-film layer coated on the sensing side. A probe is typically made of a material suitable for BLI detection such as glass. A probe has a distal end and a proximal end. The proximal end (also refers to probe tip in the application) has a sensing surface coated with a thin layer of analyte-binding molecules.

A “waveguide” refers to a device (e.g., a duct, coaxial cable, or optic fiber) designed to confine and direct the propagation of electromagnetic waves (as light).

The invention provides methods to determine DNA concentrations in viruses and determine empty vs. full virus capsids ratio. The initial step is to capture specific virus by capturing a defined amount of capsid particles on the surface of a first solid phase. The first solid phase is designed for specific virus capture with negligible non-specific binding from interfering substances in samples. The second step is to lyse the capsid to release the virus DNA from the first solid phase into a lysis solution. After separating the lysis solution from the first solid phase, the third step is to contact the lysis solution with a second solid surface. The second solid phase captures and measures the total amount of DNA derived from the captured capsid. Since the amount of capsid particles is known in the initial capture step and/or is consistent from sample to sample, the total DNA assay signal is proportional to the E/F ratio.

In one method, bio-layer interferometry (BLI) technique is used for detection. BLI is an optical technique for measuring macromolecular interactions by analyzing interference patterns of white light reflected from the surface of a biosensor tip. The method features three basic steps to determine DNA concentration in DNA virus such as AAVs and determine empty vs. full capsid ratio. The initial step is the specific virus capture of a defined amount of capsid particles on a solid phase such as the tip of a first biosensor probe. The first probe is designed for specific virus capture with negligible non-specific binding from interfering substances in samples. The second step is lysis of the capsid to release the viral DNA from the first solid phase into a lysis solution. After removing the first solid phase or the first probe, the third step is immersion of a second biosensor probe tip into the lysis solution. The second biosensor probe tip measures total DNA derived from the captured capsid. Since the amount of capsid particles is known in the initial capture step and is consistent from sample to sample, the total DNA assay signal is proportional to the E/F ratio.

In another method, fluorescent technique is used for detection. After virus capture and lysis of the captured virus, the lysed DNA is captured by a second solid phase and detected by fluorescent signals.

### Biosensor Interferometer Systems

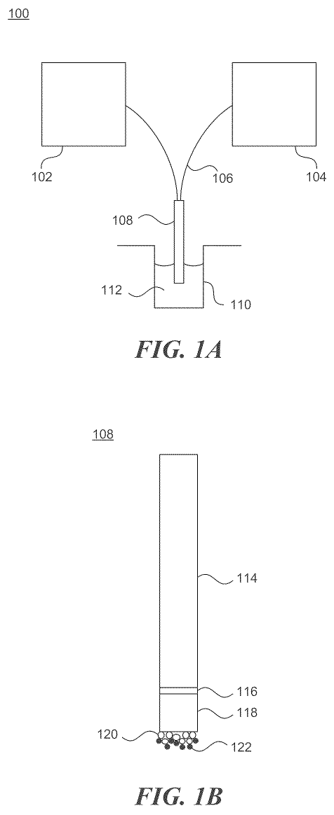

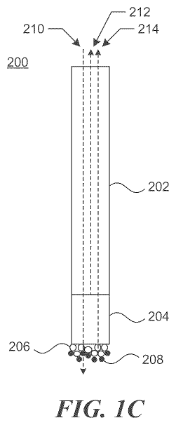

FIG. 1A illustrates one example of a biosensor interferometer system suitable for the present method. FIG. 1A depicts a biosensor interferometer 100 (or simply “interferometer”) that includes a light source 102, a detector 104, a waveguide 106, and an optical assembly 108 (also referred to as a “probe”). The probe 108 may be connected to the waveguide 106 via a coupling medium.

The light source 102 may emit white light that is guided toward the probe 108 by the waveguide 106. For example, the light source 102 may be a light-emitting diode (LED) that is configured to produce light over a range of at least 50 nanometers (nm), 100 nm, or 150 nm within a given spectrum (e.g., 400 nm or less to 700 nm or greater). Alternatively, the interferometer 100 may employ a plurality of light sources having different characteristic wavelengths, such as LEDs designed to emit light at different wavelengths in the visible range. The same function could be achieved by a single light source with suitable filters for directing light with different wavelengths onto the probe 108.

The detector 104 is preferably a spectrometer, such as an Ocean Optics USB4000, that is capable of recording the spectrum of interfering light received from the probe 108. Alternatively, if the light source 102 operates to direct different wavelengths onto the probe 108, then the detector 104 can be a simple photodetector capable of recording intensity at each wavelength. In another embodiment, the detector 104 can include multiple filters that permit detection of intensity at each of multiple wavelengths.

The waveguide 106 can be configured to transport light emitted by the light source 102 to the probe 108, and then transport light reflected by surfaces within the probe 108 to the detector 104. In some embodiments the waveguide 106 is a bundle of optical fibers (e.g., single-mode fiber optic cables), while in other embodiments the waveguide 106 is a multi-mode fiber optic cable.

As shown in FIG. 1B, the probe 108 includes a monolithic substrate 114, a thin-film layer (also referred to as an “interference layer”), and a biomolecular layer (also referred to as a “biolayer”) comprised of analyte molecules 122 that have bound to analyte-binding molecules 120. The monolithic substrate 114 is comprised of a transparent material through which light can travel. The interference layer is also comprised of a transparent material. When light is shone on the probe 108, the proximal surface of the interference layer may act as a first reflecting surface and the biolayer may act as a second reflecting surface. As further described below, light reflected by the first and second reflecting surfaces may form an interference pattern that can be monitored by the interferometer 100.

The interference layer normally includes multiple layers that are combined in such a manner to improve the detectability of the interference pattern. For example, the interference layer is comprised of a tantalum pentoxide (Ta₂O₅) layer 116 and a silicon dioxide (SiO2) layer 118. The tantalum pentoxide layer 116 may be thin (e.g., on the order of 10-40 nm) since its main purpose is to improve reflectivity at the proximal surface of the interference layer. Meanwhile, the silicon dioxide layer 118 may be comparatively thick (e.g., on the order of 650-900 nm) since its main purpose is to increase the distance between the first and second reflecting surfaces.

To illustrate a simple interferometry test, the probe 108 can be suspended in a well 110 that includes a sample 112. Analyte molecules 122 will bind to the analyte-binding molecules 120 along the distal end of the probe 108 over the course of the diagnostic test, and these binding events will result in an interference pattern that can be observed by the detector 104. The interferometer 100 can monitor the thickness of the biolayer formed along the distal end of the probe 108 by detecting shifts in a phase characteristic of the interference pattern.

FIG. 1C illustrates another biosensor interferometer probe. The probe includes a monolithic substrate that has a first and a second surfaces arranged substantially parallel to one another at opposite ends of the monolithic substrate, an interference layer coated on the second surface of the monolithic substrate, and a layer of analyte-binding molecules coated on the interference layer. The interference layer will generally be comprised of magnesium fluoride (MgF₂). A first interface between the monolithic substrate and the interference layer acts as a first reflecting surface when light is shone on the interferometric sensor, while a second interface between a biolayer formed by analyte molecules in a sample binding to the analyte-binding molecules and a solution containing the sample acts as a second reflecting surface when the light is shone on the probe. As described above, the thickness of the biolayer can be estimated based on the interference pattern of light reflected by the first and second reflecting surfaces.

The probe 200 includes an interference layer 204 that is secured along the distal end of a monolithic substrate 202. Analyte-binding molecules 206 can be deposited along the distal surface of the interference layer 204. Over the course of a biochemical test, a biolayer will form as analyte molecules 208 in a sample bind to the analyte-binding molecules 206.

As shown in FIG. 1C, the monolithic substrate 202 has a proximal surface (also referred to as a “coupling side”) that can be coupled to, for example, a waveguide of an interferometer and a distal surface (also referred to as a “sensing side”) on which additional layers are deposited. Generally, the monolithic substrate 202 has a length of at least 3 millimeters (mm), 5 mm, 10 mm, or 15 mm. In a preferred embodiment, the aspect ratio (length-to-width) of the monolithic substrate 202 is at least 5 to 1. In such embodiments, the monolithic substrate 202 may be said to have a columnar form. The cross section of the monolithic substrate 202 may a circle, oval, square, rectangle, triangle, pentagon, etc. The monolithic substrate 202 preferably has a refractive index that is substantially higher than the refractive index of the interference layer 204, such that the proximal surface of the interference layer 204 effectively reflects light directed onto the probe 200. The preferred refractive index of the monolithic substrate may be higher than 1.5, 1.8, or 2.0. Accordingly, the monolithic substrate 202 may be comprised of a high-refractive-index material such as glass (refractive index of 2.0) rather than a low-refractive-index material such as quartz (refractive index of 1.46) or plastic (refractive index of 1.32-1.49).

The interference layer 204 is comprised of at least one transparent material that is coated on the distal surface of the monolithic substrate 202. These transparent material(s) are deposited on the distal surface of the monolithic substrate 202 in the form of thin films ranging in thickness from fractions of a nanometer (e.g., a monolayer) to several micrometers. The interference layer 204 may have a thickness of at least 500 nm, 700 nm, or 900 nm. An exemplary thickness is between 500-5,000 nm (and preferably 800-1,200 nm). Here, for example, the interference layer 204 has a thickness of approximately 900-1,000 nm, or 940 nm.

In contrast to conventional probes, the interference layer 204 has a substantially similar refractive index as the biolayer. This ensures that the reflection from the distal end of the probe 200 is predominantly due to the analyte molecules 208 rather than the interface between the interference layer 204 and the analyte-binding molecules 206. In some embodiments, the interference layer 204 is comprised of magnesium fluoride (MgF₂), while in other embodiments the interference layer 204 is comprised of potassium fluoride (KF), lithium fluoride (LiF), sodium fluoride (NaF), lithium calcium aluminum fluoride (LiCaAlF₆), sodium aluminum fluoride (Na₃AlF₆), strontium fluoride (SrF₂), aluminum fluoride (AlF₃), sulphur hexafluoride (SF₆), etc. Magnesium fluoride has a refractive index of 1.38, which is substantially identical to the refractive index of the biolayer formed along the distal end of the probe 200. For comparison, the interference layer of conventional probes is normally comprised of silicon dioxide, and the refractive index of silicon dioxide is approximately 1.4-1.5 in the visible range. Because the interference layer 204 and biolayer have similar refractive indexes, light will experience minimal scattering as it travels from the interference layer 204 into the biolayer and then returns from the biolayer into the interference layer 204.

In one embodiment, the probe 200 includes an adhesion layer that is deposited along the distal surface of the interference layer 204 affixed to the monolithic substrate 202. The adhesion layer may be comprised of a material that promotes adhesion of the analyte-binding molecules 206. One example of such a material is silicon dioxide. The adhesion layer is generally very thin in comparison to the interference layer 204, so its impact on light traveling toward, or returning from, the biolayer will be minimal. For example, the adhesion layer 310 may have a thickness of approximately 3-10 nm, while the interference layer 304 may have a thickness of approximately 800-1,000 nm. The biolayer formed by the analyte-binding molecules 306 and analyte molecules 308 will normally have a thickness of several nm.

When light is shone on the probe 200, the proximal surface of the interference layer 204 may act as a first reflecting surface and the distal surface of the biolayer may act as a second reflecting surface. The presence, concentration, or binding rate of analyte molecules 208 to the probe 200 can be estimated based on the interference of beams of light reflected by these two reflecting surfaces. As analyte molecules 208 attach to (or detach from) the analyte-binding molecules 206, the distance between the first and second reflecting surfaces will change. Because the dimensions of all other components in the probe 200 remain the same, the interference pattern formed by the light reflected by the first and second reflecting surfaces is phase shifted in accordance with changes in biolayer thickness due to binding events.

In operation, an incident light signal 210 emitted by a light source is transported through the monolithic substrate 202 toward the biolayer. Within the probe 200, light will be reflected at the first reflecting surface resulting in a first reflected light signal 212. Light will also be reflected at the second reflecting surface resulting in a second reflected light signal 214. The second reflecting surface initially corresponds to the interface between the analyte-binding molecules 206 and the sample in which the probe 200 is immersed. As binding occurs during the biochemical test, the second reflecting surface becomes the interface between the analyte molecules 208 and the sample.

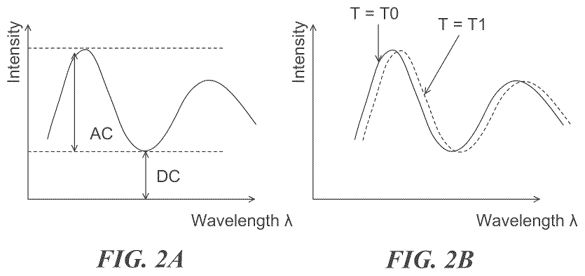

The first and second reflected light signals form a spectral interference pattern, as shown in FIGS. 2A-2B. When analyte molecules bind to the analyte-binding molecules on the distal surface of the interference layer, the optical path of the second reflected light signal will lengthen. As a result, the spectral interference pattern shifts from T0 to T1 as shown in FIG. 2B. By measuring the phase shift after a period of time, the association of an analyte molecule to an analyte-binding molecule immobilized on the distal surface of the interference layer 204 can be used to calculate analyte concentration in the sample. By measuring the phase shift continuously in real time, a kinetic binding curve can be plotted as the amount of shift versus the time. The association rate of an analyte molecule to an analyte-binding molecule immobilized on the distal surface of the interference layer can be used to calculate analyte concentration in the sample. Hence, the measure of the phase shift is the detection principle of a thin-film interferometer.

### Fluorescent Detection System

In one embodiment, a fluorescent detection system as described in U.S. Pat. No. 8,492,139, is used for measuring a fluorescent signal on a probe tip. The system comprises: (a) a probe having an aspect ratio of length to width at least 5 to 1, the probe having a first end and a second end, the second end having a sensing surface bound with a fluorescent label; (b) a light source for emitting excitation light directly to the probe's sensing surface; (c) a collecting lens pointed toward the sensing surface; and (d) an optical detector for detecting the emission fluorescent light; where the collecting lens collects and directs the emission fluorescent light to the optical detector.

The probe can be a monolithic substrate or an optical fiber. The probe can be any shape such as rod, cylindrical, round, square, triangle, etc., with an aspect ratio of length to width of at least 5 to 1, preferably 10 to 1. Because the probe is dipped in a sample solution and one or more assay solutions during an immunoassay, it is desirable to have a long probe with an aspect ratio of at least 5 to 1 to enable the probe tip's immersion into the solutions.

Any light source that can emit proper excitation light for the fluorescent label is suitable for the present invention. A prefer light source is a laser that can emit light with wavelengths suitable for fluorescent labels. For example, the laser center wavelength is preferred to be 649 nm for Cy5 fluorescent dye. A suitable optical detector for detecting emission light is a photomultiplier tube (PMT), a charge coupled device (CCD), or a photodiode.

The light source and the optical detector including the collecting lens are mounted on the same side of the probe tip surface (the sensing surface). If the sensing surface faces down, they are both mounted below the tip surface. If the sensing surface faces up, they are both mounted above the tip surface. They are closer to the sensing surface than the other end of the probe. The sensing surface is always within the numeric aperture of the collecting lens. The probe can be, but it does not have to be centrally aligned with the collecting lens.

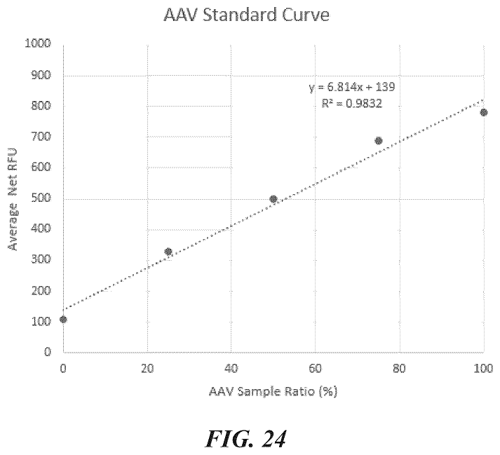

FIG. 24 of U.S. Pat. No. 8,492,139 illustrates one embodiment of the fluorescent detection system.

### Methods for Determining DNA Concentration in DNA Viruses by Bio-Layer Interferometry

The present invention is directed to a method of determining DNA concentration in DNA viruses such as adeno-associated viruses (AAVs), adenovirus, and herpes simplex virus type 1 (HSV-1), by bio-layer interferometry.

### First Aspect

In the first aspect, the method comprises the steps of: (a) obtaining a first probe having a fixed amount of anti-virus antibody immobilized on the tip of the probe, wherein the virus is AAV, adenovirus, or HSV-1; (b) dipping the first probe tip in a sample solution comprising a virus sample to capture the virus on the probe in a defined binding condition; (c) dipping the first probe in a wash solution to wash the first probe tip; (d) dipping the first probe in a lysis solution and lysing the virus from the first probe tip to obtain single-stranded DNA (ssDNA), and then removing the first probe from the lysis solution; (e) dipping a second probe comprising a first protein on the probe tip to the lysis solution, wherein the first protein binds to ssDNA; and (f) determining the ssDNA concentration in the sample by measuring the wavelength shift due to light interference, and quantitating the wavelength shift against a calibration curve to determine the ssDNA concentration;

### Second Aspect

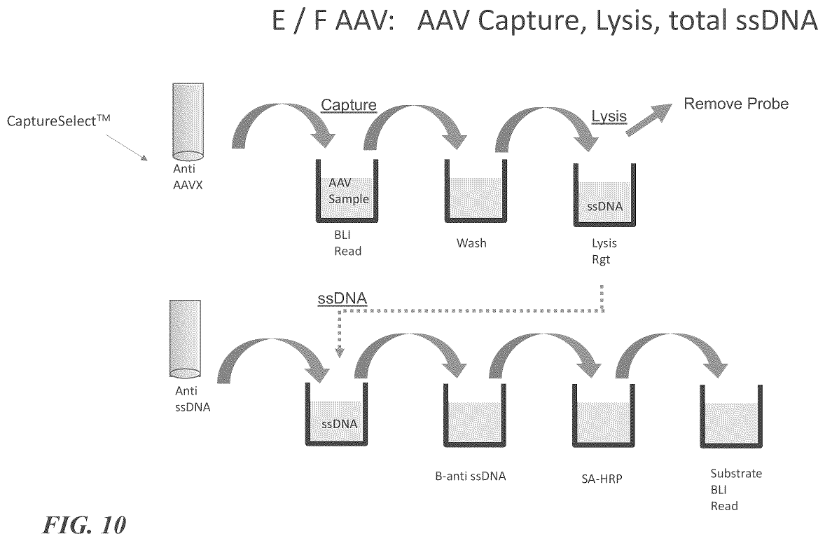

In a second aspect, the method is similar to first aspect except that after step (e), the method comprises additional steps to amplify the wavelength shift. In the second aspect, the method comprises the steps of: (a) obtaining a first probe having a fixed amount of anti-virus antibody immobilized on the tip of the probe, wherein the virus is AAV, adenovirus, or HSV-1; (b) dipping the first probe tip in a sample solution comprising a virus sample to capture the virus on the probe in a defined binding condition; (c) dipping the first probe in a wash solution to wash the first probe tip; (d) dipping the first probe in a lysis solution and lysing the virus from the first probe tip to obtain single-stranded DNA (ssDNA), and then removing the first probe from the lysis solution; (e) dipping a second probe comprising a first protein on the probe tip to the lysis solution, wherein the first protein binds to ssDNA; (f) dipping the second probe to a reagent solution comprising biotin-labelled second protein, wherein the second protein binds to ssDNA; (g) dipping the second probe in a conjugate solution comprising a conjugate comprising streptavidin and horse radish peroxidase (HRP), to bind the conjugate to the second probe; (h) dipping the second probe in a HRP substrate solution to bind the substrate to the HRP in the conjugate for a period of time; and (g) determining the ssDNA concentration in the sample by measuring the wavelength shift due to light interference, and quantitating the wavelength shift against a calibration curve to determine the ssDNA concentration; FIG. 10 illustrated one embodiment of the invention.

In step (a) and (e) of the present method, a second probe that has a tip for binding an analyte is obtained. The tip of the second probe for detecting ssDNA may have a smaller surface area with a diameter≤5 mm, preferably ≤2 mm or ≤1 mm. The small surface of the probe tip provides several advantages. In solid phase immunoassays, a probe having a small surface area has less non-specific binding and thus produces a lower background signal. Further, the reagent or sample carried over on the probe tip is extremely small due to the small surface area of the tip. This feature makes the probe tip easy to wash and results in negligible contamination in the wash solution since the wash solution has a larger volume. Negligible contamination of the wash solution and small consumption of the reagents enable the reagents and the wash solution to be re-used many times, if desired, for example, 3-10 times, 3-15 times, or 3-20 times.

Methods to immobilize a protein such as an antibody to a solid phase (the sensing surface of the probe tip) are common in immunochemistry. A protein can bind directly to the solid phase through adsorption or it can bind indirectly to the solid phase through a binding pair. For example, the probe surface can be coated with a first member of the binding pair (e.g. anti-hapten), and an anti-virus antibody labelled with a second member of the binding pair (e.g., hapten) is immobilized on the probe through the biotin-streptavidin binding.

In step (b) of the method, the first probe tip is dipped in a sample solution comprising an AAV sample for a first period of time to capture a defined amount of viruses on the probe in a defined binding condition. The binding time is from 10 minutes, 20 minutes, or 30 minutes to 1 or 2 hours. For example, the binding time is from 10 minutes to 1 hour. In a preferred embodiment, the amount of the anti-virus on the probe is fixed and the binding condition is fixed, about the same amount of viruses is capture in different samples. In general, viruses in the sample exceed the maximum binding capacity of anti-virus on the probe. In one embodiment, the virus binding is allowed to approach the maximum binding with the anti-virus on the probe.

This step provides an important aspect of the invention that the capture of AAV is standardized to the same number of viral particles from sample to sample.

In step (c), the first probe is dipped in a wash solution comprising an aqueous solution preferably having pH of 6.0-8.5 for a period of time (e.g., 5 seconds to 5 minutes, 10 seconds to 2 minutes, or 15 seconds to 1 minute). The aqueous solution can be water or a buffer having pH between 6.0 to 8.5. Preferably, the aqueous solution contains 1-10 mM or 1-100 mM of phosphate buffer, tris buffer, citrate buffer or other buffer suitable for pH between 6.0-8.5.

In step (d) of the method, the first probe tip is dipped into a lysis solution, and the lysis solution is heated to remove the DNA from the capsids, release the DNA into the lysis reagent, and denature the DNA into single strands. There are different methods to lyse DNA viruses and to release DNA that can be used in this invention; heating plus detergent lysis is preferred due to its speed. The lysis solution in general contains a detergent having a concentration at least 1% or 2%. For example, the lysis solution contains 2-4% of Tween 20. The heating step is typically at a temperature 50-90° C., or 70-90° C., or 80-95° C. for ssDNA (e.g., AAV), and 80-95° C. or 90-100° C. for dsDNA. The heating time is typically 2-5 minutes or 2-10 minutes. After the DNA is lysed from the first probe, the first probe is removed from the lysis solution.

In step (e), a second probe comprising a first protein on the probe tip is dipped in the lysis solution for a period of time (e.g., 5-60 min), wherein the first protein binds to ssDNA.

The second probe is then optionally washed in a wash solution as described in step (c).

In step (f), the second probe is dipped in a reagent solution comprising biotin-labelled second protein for a period of time (e.g., 30 seconds to 5 minutes), wherein the second protein binds to ssDNA.

The first protein and the second protein both bind to ssDNA, and they can be the same or different. For example, the first protein and the second protein are independently an antibody against ssDNA or single-stranded DNA binding protein (SSB).

The second probe is then optionally washed in a wash solution as described in step (c).

In step (g), the second probe is dipped in a conjugate solution comprising a high molecular conjugate comprising streptavidin and horse radish peroxidase (HRP) for a period of time (e.g., 10 seconds to 5 minutes, 15 seconds to 1 minute, or 20 seconds to 2 minutes), to bind the conjugate to the second probe.

In general, the high molecular weight conjugate has a molecular weight of at least 200,000, or at least 400,000, or at least 500,000, or at least 1 million Daltons, or at least 2 million Daltons. For example, the conjugate has a molecular weight of 200,000 to 10 million Daltons, 500,000 to 10 million Daltons, 1 to 10 million Daltons, or 2-10 million Daltons.

In one embodiment, the high molecular weight conjugate comprises streptavidin and crosslinked HRP, wherein the conjugate comprises at least 2, or at least 4, or at least 5, or at least 10 HRPs.

In one embodiment, the high molecular weight conjugate comprises streptavidin and HRP, where streptavidin and HRP are crosslinked together. The conjugate comprises at least 2, or at least 4, at least 5, or at least 10 HRPs, and the molar ratio of streptavidin to HRP is between about 1:2 to about 2:1, or about 1:1.5 to about 1.5 to 1, e.g., about 1:1.

In a preferred embodiment, the high molecular weight conjugate comprises streptavidin, HRP and a polymer. In the conjugate, streptavidin and HRP may bind to the polymer. The polymer in general has a molecular weight of 200,000 to 10,000,000 Daltons, or 500,000 to 10,000,000 Daltons, or 1 million to 10 million Daltons, or 2 million to 10 million Daltons. The polymer can be a polysaccharide (e.g., dextran, amylose, polysucrose), a dendrimer, or a polyethylene glycol. In one preferred embodiment, the polymer is FICOLL® (copolymers of sucrose and epichlorohydrin). The conjugate comprises at least 2, or at least 4, at least 5, or at least 10 HRPs, and the molar ratio of streptavidin to HRP is between about 1:2 to about 2:1, or about 1:1.5 to about 1.5 to 1, e.g., about 1:1.

After binding, the second probe is then optionally washed in a wash solution as described in step (c) for 1-3 times.

In step (h), the second probe is dipped in a HRP substrate solution to bind the substrate to the HRP in the conjugate for a period of time (e.g., 30 seconds to 5 minutes).

When the product of the HRP/substrate reaction binds to the surface of the optical layer, the optical layer increases its thickness and produces a nanometer wavelength shift due to light interference, and the wavelength shift is proportional to the amount of HRP on the probe. Any HRP substrate whose product binds to solid phase surfaces is suitable for the present invention. A precipitating substrate is preferred for BLI assay. Suitable substrates include 3,3′-diaminobenzidine tetrahydrochloride (DAB), 3,3′,5,5′-tetramethylbenzidine (TMB), benzamidine, 4-chloro-1-naphthol, nitro-blue tetrazolium chloride and 5-bromo-4-chloro-3′-indolyphosphate p-toluidine salt. Preferred substrates are chloronapthol, benzamidine, and tetramethylbenzidine, and chloronapthol is more preferred.

In step (i), the ssDNA concentration in the sample is determined by measuring the wavelength shift due to light interference, and the wavelength shift is quantitated against a calibration curve to determine the ssDNA concentration. The phase shift can be monitored either kinetically or determined by the difference between starting time point (T0) and end time point (T1) (see FIG. 2B).

In the above binding and washing steps, the reaction is optionally accelerated by agitating or mixing the solution in the vessel. For example, a flow such as a lateral flow or an orbital flow of the solution across the probe tip can be induced in one or more reaction vessels, including sample vessel, reagent vessel, wash vessels, and conjugate vessel, to accelerates the binding reactions, disassociation. For example, the reaction vessels can be mounted on an orbital shaker and the orbital shaker is rotated at a speed at least 50 rpm, preferably at least 200 rpm or at least 500 rpm, such as 50-200 or 500-1,500 rpm. Additionally, the probe tip can be moved up and down and perpendicular to the plane of the orbital flow, at a speed of 0.01 to 10 mm/second, in order to induce additional mixing of the solution above and below the probe tip.

In one embodiment, the second probe (anti-DNA probe) is merely dipped into the lysed sample to bind the DNA. No reagents are required to add to the lysed sample. After the binding step, the anti-DNA probe is moved to another vessel to perform the rest of the assay. The amount of DNA that is bound to the probe is a very small percentage of DNA in the sample, and therefore, the composition and content of the sample is not altered. This creates an option to use the lysed sample for other analysis. Because the sample is affinity purified by the first anti-virus probe, there is little protein remaining in the purified sample, and a subsequent PCR is easy to perform. Users may elect to determine specific sequences in addition to total DNA.

### A Third Aspect

In the third aspect, the method is similar to the method of the second aspect except that the second probe is dipped in a reagent solution comprising HRP-labelled second protein that binds to ssDNA.

In the third aspect, the method of determining DNA concentration in viruses comprises the steps of: (a) obtaining a first probe having a fixed amount of anti-virus antibody immobilized on the tip of the probe, wherein the virus is AAV, adenovirus, or HSV-1; (b) dipping the first probe tip in a sample solution comprising a virus sample to capture the virus on the probe in a defined binding condition; (c) dipping the first probe in a wash solution to wash the first probe tip; (d) dipping the first probe in a lysis solution and lysing the virus from the first probe tip to obtain single-stranded DNA (ssDNA), and then removing the first probe from the lysis solution; (e) dipping a second probe comprising a first protein on the probe tip in the lysis solution, wherein the first protein binds to ssDNA; (f) dipping the second probe in a reagent solution comprising HRP-labelled second protein, wherein the diameter of the tip surface of the second probe is ≤5 mm and the second protein binds to ssDNA; (g) dipping the second probe in a HRP substrate solution to bind the substrate to the HRP bound on the second probe for a period of time; and (h) determining the ssDNA concentration in the sample by measuring the wavelength shift due to light interference, and quantitating the wavelength shift against a calibration curve to determine the ssDNA concentration.

In the method, the first protein and the second protein are independently an antibody against ssDNA or single-stranded DNA binding protein (SSB).

In the second aspect of the method, Steps (a)-(e) are identical to those described in the first aspect.

In step (f), the second probe is dipped in a conjugate solution comprising a HRP-labelled-second protein that binds to DNA for a period of time (e.g., 10 seconds to 5 minutes, 15 seconds to 1 minute, or 20 seconds to 2 minutes), to bind the HRP-second protein conjugate to the second probe.

After binding, the second probe is then optionally washed in a wash solution as described in step (c) for 1-3 times.

In step (g), the second probe is dipped in a HRP substrate solution to bind the substrate to the HRP bound on the second probe for a period of time (e.g., 30 seconds to 5 minutes). The HRP substrate is described in the first aspect of the method.

In step (h), the ssDNA concentration in the sample is determined by measuring the wavelength shift due to light interference as described in the in the first aspect of the method.

The common reagents and common procedures in the first and the second aspects of the method are similar.

Method for Determining Empty vs. Full Virus Capsids by Bio-Layer Interferometry

The present invention is directed to a method for measuring the percentage of full virus (e.g., AAV) capsid in a virus (e.g., AAV) sample; also called measuring empty (empty virus capsid) vs. full (full virus capsid).

### A First Aspect

The BLI signal of capturing DNA viruses (e.g., AAVs) is directly proportional to the number of capsids captured in real time. In theory, if the standards and samples have the same virus (e.g., AAV) concentration or the same number of capsids captured, the BLI signals of virus binding are the same, and the ssDNA signals are proportional to the empty/full capsid ratio and the empty/full capsid ratio of a virus (e.g., AAV) sample can be determined from the standard curve, in which calibrators comprises mixtures of empty and full capsids of different ratios. The calibrators are run at the same virus concentration.

In a first aspect, the method comprises first determining the ssDNA concentration in a virus sample according to the method described above, and then converting the ssDNA concentration to % of full virus capsid using a calibration curve having DNA concentration plotted against % of full virus capsid standards. The calibration curve can be established with results of calibrators that run together with unknown samples, or with results of calibrators from a separate run.

The invention is an easy and accurate method to determine virus (e.g., AAV) transgene empty/full ratios. The method uses two probes (anti-virus probe and anti-ssDNA probe) and comprises three key steps: virus capture, lysis, and total ssDNA quantitation. The anti-AAV BLI probe of the present invention demonstrates a reproducible and high precision capture of AAVs that can be monitored in real time. The binding of AAV to the anti-AAV sensor can be detected by BLI without the use of a label.

Performing both AAV capture and ssDNA quantitation steps with BLI instrument and automated protocols is convenient.

### A Second Aspect (Normalization)

If the standards and samples have different virus concentrations or different numbers of capsids captured, the BLI signals of virus (e.g., AAV) binding may be different; which may then bias the data analysis in the ssDNA assay to derive empty/full ratio in a virus sample. The present invention provides a method to normalize the BLI signals of virus binding and align the sample concentration difference in order to determine the empty capsid/full capsid ratio.

In using normalization for the empty/full capsid ratio determination, the virus binding signal in each sample is normalized by comparison with the virus binding signal of one or more calibrators to give a normalization factor. The ssDNA signal (wavelength) of a sample is multiplied by the normalization factor to yield the normalized wavelength shift value of a sample. The calibrators are run at the same virus concentration and are used to generate a standard curve of BLI signal vs. empty/full virus capsid ratio. DNA virus samples are run together with the calibrators and the wavelength shift values of a sample is normalized by the normalization factor, then the empty/full ratio of the sample is determined from the standard curve. As the normalized wavelength shift values take into account the capture difference due to different virus concentration in samples, the normalization may be more accurate to calculate the empty/full ratio in some situation.

In first embodiment, the method comprises the steps of: (a) obtaining a first probe having a fixed amount of anti-DNA virus (e.g., AAV) antibody immobilized on the tip of the probe; (b) dipping the first probe tip in a sample solution comprising a DNA virus sample to capture AAV on the probe in a defined binding condition for a period of time to measure a first wavelength shift due to light interference as a result of the binding virus on the probe; (c) determining a normalization factor based on the first wavelength shift of the sample in comparison with (i) the first wavelength shift of a calibrator, or (ii) the average of the first wavelength shifts of all calibrators; (d) dipping the first probe in a wash solution to wash the first probe tip; (e) dipping the first probe in a lysis solution and lysing the AAV from the first probe tip to obtain single-stranded DNA (ssDNA), and then removing the first probe from the lysis solution; (f) dipping a second probe comprising a first protein on the probe tip in the lysis solution, wherein the first protein binds to ssDNA; (g) dipping the second probe in a reagent solution comprising biotin-labelled second protein, wherein the second protein binds to ssDNA; (h) dipping the second probe in a conjugate solution comprising a conjugate comprising streptavidin and horse radish peroxidase (HRP), to bind the conjugate to the second probe; (i) dipping the second probe in a HRP substrate solution to bind the substrate to the HRP in the conjugate for a period of time; and measuring the second wavelength shift due to light interference; (j) applying the normalization factor to the second wavelength shift to produce a normalized wavelength shift, and (k) quantitating the normalized wavelength shift against a calibration curve having normalized wavelength shift plotted against % of full AAV capsids to determine the percentage of full AAV capsids in the sample. The first protein and the second protein are independently SSB or anti-DNA antibody. The first protein and the second protein can be the same or different.

In this method, the details of steps (a), (b), (d)-(i) are similar to the corresponding steps described above in a method of determining DNA concentration.

In step (c), the normalization factor is determining by comparing the first wavelength shift of virus binding to the probe of the sample with that of one or more calibrators. A calibrator is a virus (e.g., AAV) sample with known empty/full capsid ratio and captured at the same virus concentration as the other calibrators. The calibrators can be run together with the samples under the same assay conditions. Alternatively, the calibrators can be run separately from the samples under the same assay conditions. In general, 1-6, 2-6, 3-6, 2-5, or 3-5 calibrators are used to establish a normalization factor. The normalization factor is determined by the ratio of the first wavelength shift of the sample to (i) the first wavelength shift of a calibrator, or (ii) the average of the first wavelength shifts of all calibrators. In one preferred embodiment, the normalization factor is determined by the ratio of the first wavelength shift of the sample to the average of the first wavelength shifts of all calibrators.

In step (i), a normalized wavelength shift of the second wavelength shift is calculated by applying the normalized factor to the second wavelength shift.

In step (j), the percentage of full virus capsids in the sample is determined by quantitating the normalized wavelength shift against a calibration curve. The calibration curve is established by measuring the normalized shifts of calibrator samples with known percentages of full virus capsids and plotting normalized wavelength shifts against percentages of full virus capsids.

Typically, the samples and calibrators are run in same run, but they can be run in different runs on the same day. Running calibrators on a different day is possible providing the samples and the instrument are stable.

The normalization procedures of this embodiment are illustrated in Example 12.

In a second embodiment, the method is similar to that of the first embodiment, except the signal detection is without the biotin-streptavidin amplification. In second embodiment, the method comprises the steps of: (a) obtaining a first probe having a fixed amount of anti-DNA virus (e.g., AAV) antibody immobilized on the tip of the probe; (b) dipping the first probe tip in a sample solution comprising a DNA virus sample to capture AAV on the probe in a defined binding condition for a period of time to measure a first wavelength shift due to light interference as a result of the binding virus on the probe; (c) determining a normalization factor based on the first wavelength shift of the sample in comparison with (i) the first wavelength shift of a calibrator, or (ii) the average of the first wavelength shifts of all calibrators; (d) dipping the first probe in a wash solution to wash the first probe tip; (e) dipping the first probe in a lysis solution and lysing the AAV from the first probe tip to obtain single-stranded DNA (ssDNA), and then removing the first probe from the lysis solution; (f) dipping a second probe comprising a first protein on the probe tip in the lysis solution, wherein the first protein binds to ssDNA; (g) dipping the second probe in a reagent solution comprising HRP-labelled second protein, wherein the second protein binds to ssDNA; (h) dipping the second probe in a HRP substrate solution to bind the substrate to the HRP bound on the second probe for a period of time and measuring the second wavelength shift due to light interference; (i) applying the normalization factor to the second wavelength shift to produce a normalized second wavelength shift, and (j) quantitating the normalized second wavelength shift against a calibration curve having normalized wavelength shift plotted against % of full AAV capsids to determine the percentage of full AAV capsids in the sample.

### High Molecular Weight HRP Conjugate

The present disclosure also provides a conjugate comprising streptavidin, HRP and crosslinked FICOLL®. The crosslinked FICOLL® has a molecular weight of at least 500,000 daltons, and preferably at least 1 million, or 2 million, or 5 million, or 10 million Daltons. The conjugate has at least 2 streptavidin and at least 2 HRPs, and preferably has at least 4 streptavidin and at least 4 HRPs. In one embodiment, the crosslinked FICOLL® has a molecular weight of 500,000-10 million Daltons, or 1-10 million Daltons, and each crosslinked FICOLL® binds at least 2 HRPs and at least 2 streptavidins. In one embodiment, the crosslinked FICOLL® has a molecular weight of at least 2 million Daltons (e.g., 1-10 million Daltons), and each crosslinked FICOLL® binds at least 4 or 5 HRPs and at least 4 or 5 streptavidin. In one embodiment, the crosslinked FICOLL® has a molecular weight of at least 2 million Daltons or at least 5 million Daltons (e.g., 2-10 million Daltons or 5-10 million Daltons), and each crosslinked FICOLL® binds at least 10 HRPs and at least 10 streptavidins.

In one embodiment, the conjugate has a molar ratio of FICOLL®:streptavidin:HRP of 1:2-10:2-10. In another embodiment, the conjugate has a molar ratio of FICOLL®:streptavidin:HRP of 1:3-7:3-7. For example, each crosslinked FICOLL® molecules binds at least 4-10 HRP and 4-10 streptavidin.

### BLI Probes

The present invention also provides a probe comprising a monolithic substrate, a thin-film layer, and a biomolecular layer immobilized on the tip of the probe, wherein the biomolecular layer comprises analyte molecules, analyte-binding molecules, HRP, and chloronapthol, wherein the diameter of the tip surface is ≤5 mm.

### Methods for Determining DNA Concentration in DNA Viruses Using Two Different Solid Surfaces and BLI for Detection

The present invention is further directed to a method of determining DNA concentration in DNA viruses such as such as AAVs, adenovirus, and HSV-1, by using two different solid surfaces for capturing virus capsids and ssDNA and BLI for detection.

The method comprises the steps of: (a) obtaining a first solid surface immobilized with a fixed amount of anti-virus antibody, wherein the virus is AAV, adenovirus, or HSV-1; (b) contacting the first solid surface with a sample solution comprising a virus sample to capture the virus on the first solid surface in a defined binding condition; (c) washing the first solid surface with a wash solution to remove the non-bound materials from the solid surface; (d) contacting the first solid surface with a lysis solution and lysing the virus from the first solid surface to obtain single-stranded DNA (ssDNA) in the lysis solution, and then collecting the lysis solution; (e) dipping a probe in the lysis solution, wherein the probe comprises a first protein on the probe tip, wherein the first protein binds to ssDNA; (f) optionally washing the probe; (g) dipping the probe in a reagent solution comprising HRP-labelled second protein, wherein the second protein binds to ssDNA; (h) dipping the probe in a HRP substrate solution to bind the substrate to the HRP bound on the probe for a period of time; and (i) determining the ssDNA concentration in the sample by measuring the wavelength shift due to light interference, and quantitating the wavelength shift against a calibration curve to determine the ssDNA concentration.

In one embodiment, the above step (g) can be substituted by steps (g1) and (g2) to amplify the signal; where (g1) is: dipping the probe in a solution comprising a second protein conjugated with a biotin, and (g2) is: dipping the probe in a solution comprising a conjugate comprising streptavidin and HRP.

In one embodiment, the anti-virus antibody is coated on the first solid surface by first immobilizing streptavidin on the solid surface and then incubating the solid surface with a biotin-anti-virus conjugate.

The first solid surface can be selected from the group consisting of: beads such as magnetic beads; tubes; microwells such as microplates containing 96 well or 384 wells in ELISA format; polystyrene beads or latex beads in micron and nanometer sizes; nanospheres; microspheres; Sepharose, agarose, dextran and other carbohydrate base resins; glass/silica, borate silicate/quartz materials in slide, pin, fiber, microsphere, or bead format; magnetic particles with latex, dextran, polymer matrices in nano, microsphere, or rod format; quantum dots nanocrystals, Europium Chelates conjugates with or without polymer coating on Europium surfaces.

In one embodiment, the first solid phase is magnetic beads. Magnetic beads have a large surface area to capture virus capsids.

In one embodiment, the probe is a glass probe with a tip surface of ≤5 mm.

### Methods for Determining DNA Concentration in DNA Viruses by Non-BLI Immunoassay

The present invention is also directed to a method of determining DNA concentration in DNA viruses such as such as AAVs, adenovirus, and HSV-1, by non-BLI solid phase immunoassays.

The method comprises the steps of: (a) obtaining a first solid surface immobilized with a fixed amount of anti-virus antibody, wherein the virus is AAV, adenovirus, or HSV-1; (b) contacting the first solid surface with a sample solution comprising a virus sample to capture the virus on the first solid surface in a defined binding condition; (c) washing the first solid surface with a wash solution to remove the non-bound materials from the solid surface; (d) contacting the first solid surface with a lysis solution and lysing the virus from the first solid surface to obtain single-stranded DNA (ssDNA) in the lysis solution, and then collecting the lysis solution; (e) contacting the ssDNA-containing lysis solution with a second solid surface immobilized with a first protein, wherein the first protein binds to ssDNA; (f) washing the second solid surface; (g) contacting the second solid surface with a second protein conjugated with a label; and (h) detecting the label and determining the DNA concentration in the virus; wherein the first protein and the second protein are independently SSB or anti-DNA antibody. The first protein and the second protein can be the same or different.

Labels (reporter molecules) suitable for the present invention are those that can generate a signal. The labels include fluorescent dyes, chemiluminescent dyes, enzymes, radiolabels, and any other reporter molecules commonly known to a person skilled in the art. Suitable fluorescent dyes include Alexa Fluor, Cyanine 5, Cyanine 3, Q-Dots, etc. Suitable chemiluminescent dyes include acridinium esters. Suitable enzymes include horse radish peroxidase (HRP), alkaline phosphatase, and beta galactosidase.

In one embodiment, the above step (g) can be substituted by steps (g1) and (g2) to amplify the signal; where (g1) is contacting the second solid surface with a second protein conjugated with a biotin, and (g2) is contacting the second solid surface with a conjugate comprising streptavidin and a label for detection.

In one embodiment, the anti-virus antibody is coated on the first solid surface by first immobilizing a first member of a binding pair on the solid surface and then incubating the solid surface with a conjugate of an anti-virus antibody and the second member of the binding pair.

The first and the second solid surface can be independently selected from the group consisting of: beads such as magnetic beads; tubes; microwells such as microplates containing 96 well or 384 wells in ELISA format; polystyrene beads or latex beads in micron and nanometer sizes; nanospheres; microspheres; Sepharose, agarose, dextran and other carbohydrate base resins; glass/silica, borate silicate/quartz materials in slide, pin, fiber, microsphere, or bead format; magnetic particles with latex, dextran, polymer matrices in nano, microsphere, or rod format; quantum dots nanocrystals, Europium Chelates conjugates with or without polymer coating on Europium surfaces.

The first and the second solid surface can be made of the same material or different material.

In one embodiment, the first and/or the second solid phase is magnetic bead.

### Methods for Determining DNA Concentration in DNA Viruses by a Fluorescent Immunoassay

The present invention is also directed to a method of determining DNA concentration in DNA viruses such as AAVs, adenovirus, and HSV-1, by fluorescent solid phase immunoassays.

The method comprises the steps of: (a) obtaining a first solid surface immobilized with a fixed amount of anti-virus antibody, wherein the virus is AAV, adenovirus, or HSV-1; (b) contacting the first solid surface with a sample solution comprising a virus sample to capture the virus on the first solid surface in a defined binding condition; (c) washing the first solid surface with a wash solution to remove the non-bound materials from the solid surface; (d) contacting the first solid surface with a lysis solution and lysing the virus from the first solid surface to obtain single-stranded DNA (ssDNA) in the lysis solution, and then collecting the lysis solution; (e) contacting the ssDNA-containing lysis solution with a second solid surface immobilized with a first protein, wherein the first protein binds to ssDNA; (f) washing the second solid surface; (g) contacting the second solid surface with a second protein conjugated with a fluorescent label; and (h) detecting the label and determining the DNA concentration in the virus; wherein the first protein and the second protein are independently SSB or anti-DNA antibody. The first protein and the second protein can be the same or different. The fluorescent label, for example, is Cy3, Cy5, AlexaFluor™ Plus 647.

In one embodiment, the above step (g) can be substituted by steps (g1) and (g2) to amplify the signal; where (g1) is contacting the second solid surface with a second protein conjugated with a biotin, and (g2) is contacting the second solid surface with a conjugate comprising streptavidin and a fluorescent label for detection.

In one embodiment, the conjugate in step (g2) is a conjugate comprising streptavidin, fluorescent molecules, and a high molecular polymer, wherein the streptavidin and the fluorescent molecules are covalently linked to a high molecular polymer to increase the loading of streptavidin and the fluorescent molecules. The high molecular weight polymer serves as a linker or spacer. The polymer in general has a molecular weight of 10,000 to 10 million Daltons, preferably 100,000 to 5 millions, 400,000 to 5 millions, or 1 to 5 millions. The polymer can be a polysaccharide (e.g., dextran, amylose), a dendrimer, or a polyethylene glycol. In one preferred embodiment, the polymer is FICOLL® (copolymers of sucrose and epichlorohydrin).

In one embodiment, the anti-virus antibody is coated on the first solid surface by first immobilizing a first member of a binding pair on the solid surface and then incubating the solid surface with a conjugate of an anti-virus antibody and the second member of the binding pair.

The first and the second solid surface can be independently selected from the group consisting of: beads such as magnetic beads; tubes; microwells such as microplates containing 96 well or 384 wells in ELISA format; polystyrene beads or latex beads in micron and nanometer sizes; nanospheres; microspheres; Sepharose, agarose, dextran and other carbohydrate base resins; glass/silica, borate silicate/quartz materials in slide, pin, fiber, microsphere, or bead format; magnetic particles with latex, dextran, polymer matrices in nano, microsphere, or rod format; quantum dots nanocrystals, Europium Chelates conjugates with or without polymer coating on Europium surfaces.

The first and the second solid surface can be made of the same material or different material.

In one embodiment, the first and/or the second solid phase is magnetic bead.

In one embodiment, the first and/or the second solid phase is a glass probe with a tip surface of ≤5 mm.

The invention is illustrated further by the following examples that are not to be construed as limiting the invention in scope to the specific procedures described in them.

### Examples

### Materials and Instrument for Examples 1-12

Recombinant Streptavidin was obtained from IBA. Horseradish peroxidase (HRP) RZ=1 was obtained from Santa Cruz BioTech. SMCC, succinimidyl 4-(N-maleimidomethyl)cyclohexane-1-carboxylate, DMF, anhydrous dimethylformamide were obtained from Thermo Scientific without further purification before usage. Cy5-NHS, Cyanine 5 monosuccinimidyl ester was ordered from ATT Bioquest. N-acetyl-1-cysteine and n-ethylmaleimde were purchased from MP Biomedicals and Thermo Scientific. 10× Phosphate Buffer Saline (Thermo Scientific) was used by 10 fold dilution with MilliQ water before use. 0.5M Carbonate Buffer, pH9.4 was used directly from BioWorld supply.

AAVX probe (Gator Bio, Inc.) was used to capture AAVs.

UV/VIS measurements were done by Spectrophotometer (Thermo BioMate 3S).

Centrifugation was done by Centrifuge (Beckman Allegra 6R). Proteins were desalted by Zeba Spin columns 2 ml size, 7K MWCO (Thermo Scientific). Bioconjugates were purified by Cytiva CL-4B Sepharose medium custom packed in XK 16×400 column connected to Cytiva AKTA Go Purification System.

Gator® Prime system (Gator Bio, Inc.) was used for BLI measurement.

### Materials and Reagents for Examples 13-16

Dynabeads MyOne Streptavidin C1, Cat #65001, 65002
Single-Stranded DNA Binding Protein (SSB), ThermoFisher Scientific, Cat #70032Z500UG
M13 Single-stranded DNA, New England Biolabs, Cat #N4040S
Anti-ssDNA/dsDNA antibody, Absolute Antibody, Cat #Ab00347-1.1
Anti-ssDNA/dsDNA-HRP conjugate, Absolute Antibody, Cat #Ab00347-1.1-HRP
Empty AAV8 Capsids, Vigene Biosciences, Cat #AV8-ET
Full AAV8 Capsids, Vigene Biosciences, Cat #CV10008
EZLabel™ Protein FITC Labeling Kit, BioVision, Cat #K832-5
Tween-20, Sigma, Cat #P1379-100 ML
TMB Substrate, Thermofisher Scientific, Cat #34022
Stop solution, 0.3M H₂SO₄
Thermo Scientific™ Pierce™ 96-Well Polystyrene white opaque microplates, Fisher Scientific, Cat #15042

### Example 1. Preparation of Crosslinked-FICOLL® 400-SPDP

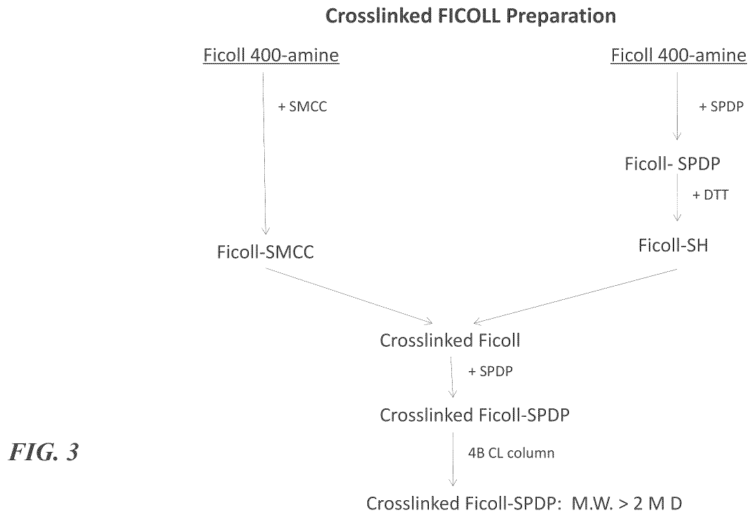

Crosslinked-FICOLL® 400-SPDP (succinimydyl 6-[3-[2-pyridyldithio]-proprionamido]hexanoate, Invitrogen) was prepared according to Example 1 of U.S. Pat. No. 9,434,789. FIG. 3 shows a flow chart of its preparation.

### Example 2. Preparation of Cyanine 5 and Maleimide Labelled Streptavidin

10 mg of Streptavidin was dissolved in 500 ul PBS buffer to make it as 20 mg/ml. 50 μl of 0.5M Carbonate Buffer was added to the Streptavidin solution. 30 μl (MCR=1) of 5 mg/ml of Cyanine 5 monosuccinimidyl ester in anhydrous dimethylformamide (DMF) was added to above streptavidin solution and let it react at room temperature for 1 hour. The Cy5 in this case served as a tracer enabling quantification of the streptavidin content in the subsequently prepared Cy5-Streptavidin/HRP-Crosslinked FICOLL® conjugate. 10 mg/ml SMCC, succinimidyl 4-(N-maleimidomethyl)cyclohexane-1-carboxylate in anhydrous dimethylformamide (DMF) was freshly prepared and 52 μl (MCR=8.7) of this was added to Cy5 labelled Streptavidin aforementioned. The SMCC labelling reaction was run at room temperature for another 1 hour. 2 ml size Zeba Spin Desalting column (7K MWCO) prepared following Thermo Scientific Instruction Guide. Cy5/SMCC dual labeled Streptavidin reaction mixture was loaded to the column and centrifuge at 1000× g for 2 min, the flow-through that contains sample was collected. The concentration of Streptavidin and Cy5 labelling ratio were estimated by 280 and 655 nm absorption by Spectrophotometer. Streptavidin is labeled with Cy5 so that the labelled streptavidin can be monitored throughout the conjugation step.

### Example 3. Preparation of Maleimide Labelled Horseradish Peroxidase

12 mg of Horseradish peroxidase (HRP) was dissolved in 600 μl 1×PBS buffer, followed with 60 ul of 0.5M Carbonate buffer addition. 72 μl of freshly prepared 10 mg/ml SMCC, succinimidyl 4-(N-maleimidomethyl)cyclohexane-1-carboxylate in anhydrous dimethylformamide (DMF) was added to the HRP solution to make up MCR=7.2 of SMCC vs HRP molar ratio. After 1 hour of room temperature reaction, the 2 ml Zeba Spin Desalting column (7K MWCO) was prepared by Thermo Scientific Instruction Guide. Reaction mixture of maleimide labelled HRP was loaded on the top of the spin column and centrifuge at 1000×g for 2 min. The purified sample was collected and the amount of maleimide labelled HRP was estimated by 280 nm absorption by Spectrophotometer.

### Example 4. Preparation of Cy5-Streptavidin, Horseradish Peroxidase, Crosslinked FICOLL® Conjugates

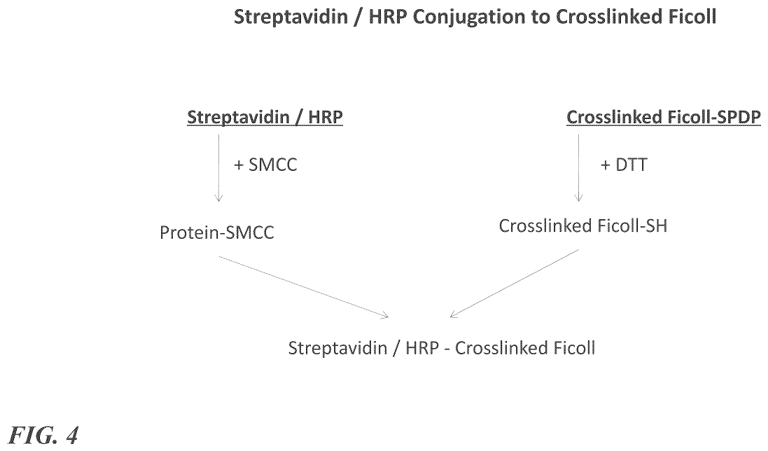

The flow chart of the streptavidin/HRP-Crosslinked FICOLL® is depicted on FIG. 4. Crosslinked FICOLL®-SPDP, (succinimidyl 3-(2-pyridyldithio)propionate), reacted with DTT, (Dithiothreitol) 38 mg/ml with 1:500 fold molar excess for 1 hour at room temperature. At the end of reaction, the mixture was loaded to a 2 ml size Zebra Spin Desalting column (7K MWCO) which was prepared by Thermo Scientific Instruction Guide. The Mercapto labelled Crosslinked FICOLL® was collected as the flow-through of the Zeba Spin column by centrifuge at 1000×g for 2 min. This sample was reacted with the Cyanine 5/maleimide labelled Streptavidin (Example 2) and maleimide labelled Horseradish peroxidase (Example 3) in the Table 1.

TABLE 1
Starting Ratio of Regents in Reaction Mixture

| Crosslinked | Cy5- | Horseradish | Molar Ratio |
|---|---|---|---|
| FICOLL ® | Streptavidin- | peroxidase- | (FICOLL ®: |
| (mg) | SMCC (mg) | SMCC (mg) | SA:HRP) |
| 1 | 1.1 | 0.8 | 1:4:5 |

The Coupling of Crosslinked FICOLL® mercaptan (1 mg) with maleimide-streptavidin (1.1 mg) and maleimide-horseradish peroxidase (0.8 mg) proceeded at room temperature for 4 hours, then quenched with n-acetyl-1-cysteine 16 mg/ml in 1×PBS (6.25 ul per mg of Streptavidin usage) and n-ethylmaleimde 32 mg/ml in 1×PBS (10 ul per mg of Streptavidin usage) sequentially. The quenched reaction mixture was purified with CL-4B column with AKTA Go purification system in 1 ml/min flow rate (1×PBS mobile phase). The first eluted peak was the desired product.

### Example 5A. Preparation of Anti-Fluorescein-Crosslinked FICOLL® Conjugates

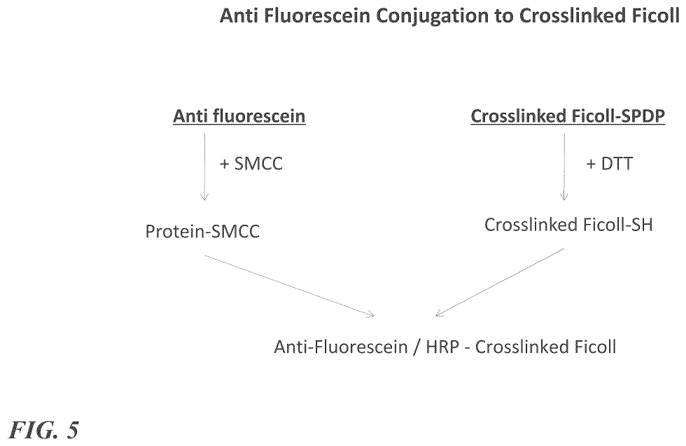

FIG. 5 presents the flow chart for the preparation of anti-fluorescein-crosslinked FICOLL®. Anti-Fluorescein (Jackson ImmunoResearch) at 1.5 mg/ml in 1 ml PBS was mixed with 1.9 ul SMCC at 5 mg/ml DMF and reacted for 1 hour at room temperature followed by purification on a PD 10 column.

The thiols on crosslinked FICOLL®-SPDP were deprotected by adding 30 ul DTT at 38 mg/ml to 0.7 mg crosslinked FICOLL® 400-SPDP in 1 ml PBS and reacting for 1 hour at room temperature followed by a PD 10 column to purify the crosslinked FICOLL®. The anti-fluorescein-SMCC was mixed with crosslinked FICOLL®-SH and reacted overnight at room temperature. 10 ul NEM (Aldrich) at 12.5 mg/ml was then added and reacted for ½ hour at room temperature. The conjugate was then purified on a Sepharose 4B CL column.

### Example 5B: Preparation of Cy5-Streptavidin-Crosslinked FICOLL®

### (a) Cy 5 Labeling of Streptavidin

32 μL of Cy 5-NHS (GE Healthcare) at 5 mg/mi in DMF reacted with 1 ml of streptavidin (Scripps Labs) at 2.4 mg/ml in 0.1 M sodium carbonate buffer pH 9.5 for 40 minutes at 30° C. Applying the mixture to a PD 10 column (Pharmacia) removed unconjugated Cy 5. Spectral analysis indicated 2.8 Cy 5 linked per streptavidin molecule.
(b) Conjugation of Cy 5-Streptavidin to Crosslinked FICOLL®

5.8 μL of SMCC (Pierce Chemical) at 10 mg/ml in DMF reacted with 2 mg Cy 5-streptavidin in 1 ml PBS pH 7.4 for 1 hour at room temperature to prepare Cy5-SA-SMCC. Applying the mixture to a PD 10 column removed unbound SMCC.

The thiols on crosslinked FICOLL® 400-SPDP (see Example 1) were deprotected by adding 30 μL DTT at 38 mg/ml to 0.7 mg crosslinked FICOLL® 400-SPDP in 1 ml PBS and reacting for 1 hour at room temperature followed by a PD 10 column to purify the crosslinked FICOLL® 400-SH.

The Cy5-SA-SMCC was mixed with crosslinked FICOLL® 400-SH and reacted overnight at room temperature. 10 μL NEM (N-ethyl-maleimide, Aldrich) at 12.5 mg/ml was then added and reacted for 30 minutes at room temperature. The Cy5-SA-crosslinked FICOLL® conjugate was then purified on a Sepharose 4B CL column.

### Example 6. Preparation of Reagents and Anti-ssDNA Probe for ssDNA Assay (BLI)

Quartz probes, 1 mm diameter and 2 cm in length, with BLI optical layer at the distal tip were coated with aminopropylsilane using a chemical vapor deposition process (Yield Engineering Systems, 1224P) following manufacturer's protocol. The probe tip was then immersed in a solution of murine monoclonal anti-fluorescein-crosslinked FICOLL® (from Example 5) at 10 μg/ml in PBS at pH 7.4. After allowing the antibody to adsorb to the probe for 15 minutes, the probe tip was washed in PBS.

The probe tip was then immersed in a solution containing fluorescein-labeled murine monoclonal anti-ssDNA (Sigma cat #MAB3868) at 10 μg/ml. The antibody was fluorescein labeled by a standard method. After 10 minutes, the probe was washed in PBS. Probes were then immersed in a solution of 15% sucrose followed by drying the probes at 37° C. for 20 minutes. Monoclonal anti-ssDNA (Sigma cat #MAB3868) was biotinylated by a standard method.

### Example 7A: ssDNA Assay (BLI)

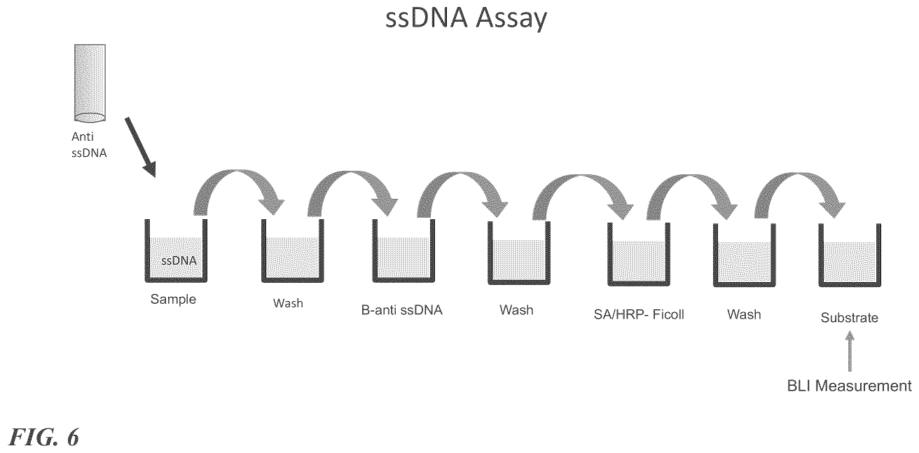

FIG. 6 illustrates the steps in performing an assay for ssDNA. The anti-ssDNA probe (Example 6) was immersed in a ssDNA sample, followed by a wash, then immersed in a B-anti-ssDNA reagent, followed by a wash, then immersed in a streptavidin/HRP-FICOLL® reagent, followed by a wash. Lastly, the probe was immersed in substrate where the bound HRP enzyme activity was monitored by BLI instrument (Gator Prime, GatorBio) according to manufacturer's protocol. A chloronapthol substrate (1.68 mM) was used. When chloronapthol bound to HRP, the optical layer increased its thickness producing a nanometer shift proportional to the amount of HRP on the probe.

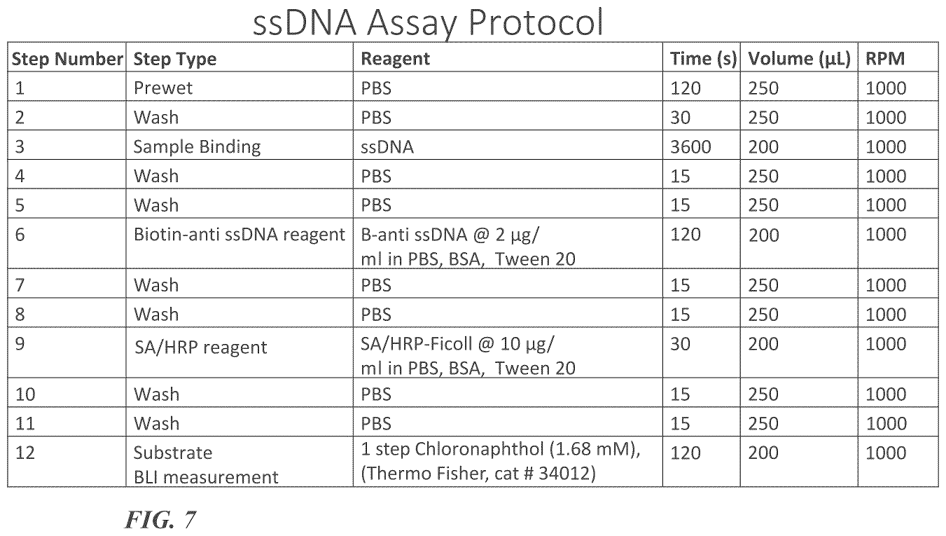

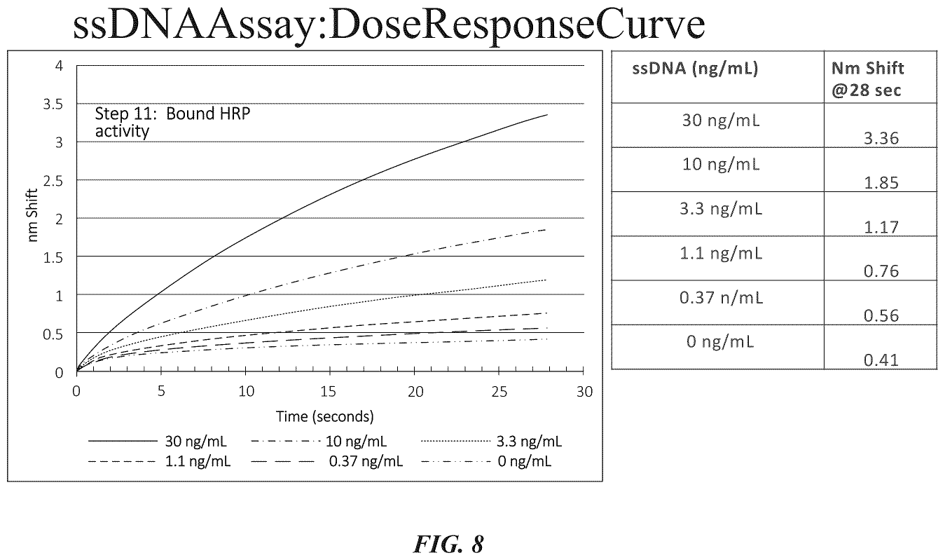

FIG. 7 shows the details in performing a ssDNA assay, i.e. reagent concentrations, volumes, etc. FIG. 8 presents the nanometer shifts with standard curve of M13 plasmid DNA (New England Biolabs, cat #N4040S) samples (see step 12 of FIG. 7), which correspond to the bound HRP enzyme activity. This ssDNA assay uses a monoclonal anti-ssDNA antibody, however, other ssDNA binding proteins can be used, such as SSB (single-stranded DNA binding protein).

### Example 7B. ssDNA Assays with Different Streptavidin/HRP Conjugates (BLI)

The ssDNA Assays were performed with the same protocols as described in Example 7A and FIG. 7, except the streptavidin (SA)/HRP reagent used was (i) SA/HRP-crosslinked FICOLL®, (ii) HRP-SA conjugate (ThermoFisher, Ultra-Streptavidin-HRP, N504, 1:1500), or (iii) streptavidin-HRP (Sigma-Aldrich, streptavidin-peroxidase polymer, S2438, 1:1000 dilution).

The results of different conjugates are shown in Table 2. The Specific DNA signal was better with the SA/HRP-FICOLL® conjugate.

TABLE 2
DNA Signals with Different Conjugates

| | Delta nm shift | Delta nm shift |
|---|---|---|
| Conjugate | DNA at 25 ng/ml | DNA at 0 ng/ml |
| SA/HRP-crosslinked FICOLL ® | 7.23 | 0.8 |
| Thermo N504 | 6.20 | 0.47 |
| Sigma S2438 | 6.57 | 0.78 |

### Example 8: Reproducibility of AAV Binding (BLI)

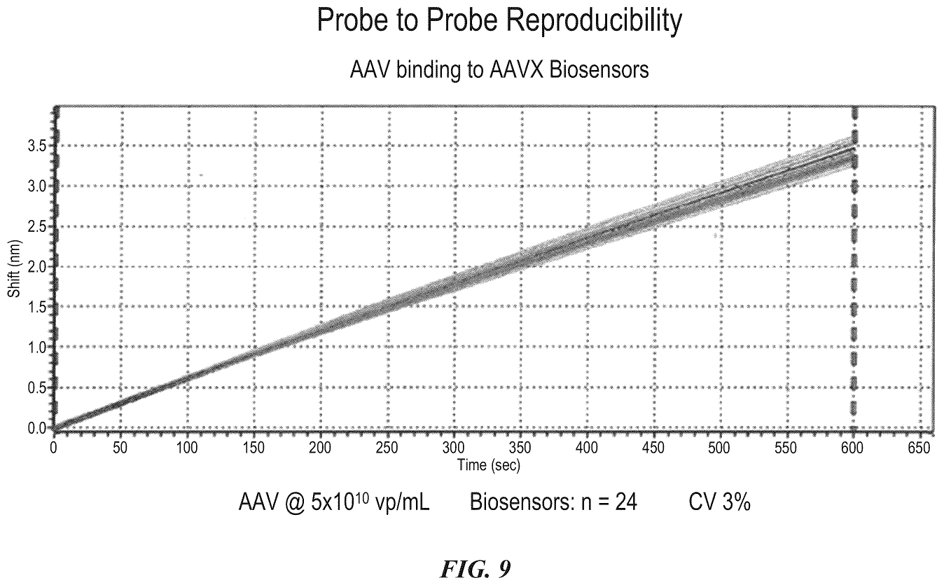

AAVX probes AAVX biosensor (GatorBio #PL168-160017), which were coated with anti-AAV, were immersed in 200 μL samples containing 5×10¹⁰ vp (viral particles)/mL of Empty AAV8 (Vigene Biosciences AV8-ET) in PBS buffer with BSA and Tween 20. The binding occurred with 1000 orbital rpm, while the nm shift was monitored in Gator Prime BLI instrument. FIG. 9 shows the wavelength shift (nm) vs. time (second) of 24 replicate probe assays. The binding was highly reproducible from probe to probe, with the coefficient of variation of 3%.

### Example 9: Empty vs. Full (E/F) Method (BLI)

FIG. 10 outlines the 3 major steps in the invention, AAV capture, lysis, then total AAV DNA measurement. The AAV capture step employs an AAVX biosensor to specifically capture AAV in the sample. The AAVX has the probe tip coated with a camelid nanobody (CapSelect™, ThermoFisher) that binds AAV. The capture is allowed to proceed until the maximum binding capacity of the probe is met. After capsid capture and a wash sequence the probe is transferred to a microwell containing a lysis reagent to release the DNA from the capsid. After the lysis step the AAVX probe is removed and an assay for ssDNA is performed as described in Example 1. Since the same number of capsids were initially captured, the ssDNA concentration in the lysed samples is proportional to the E/F ratio.

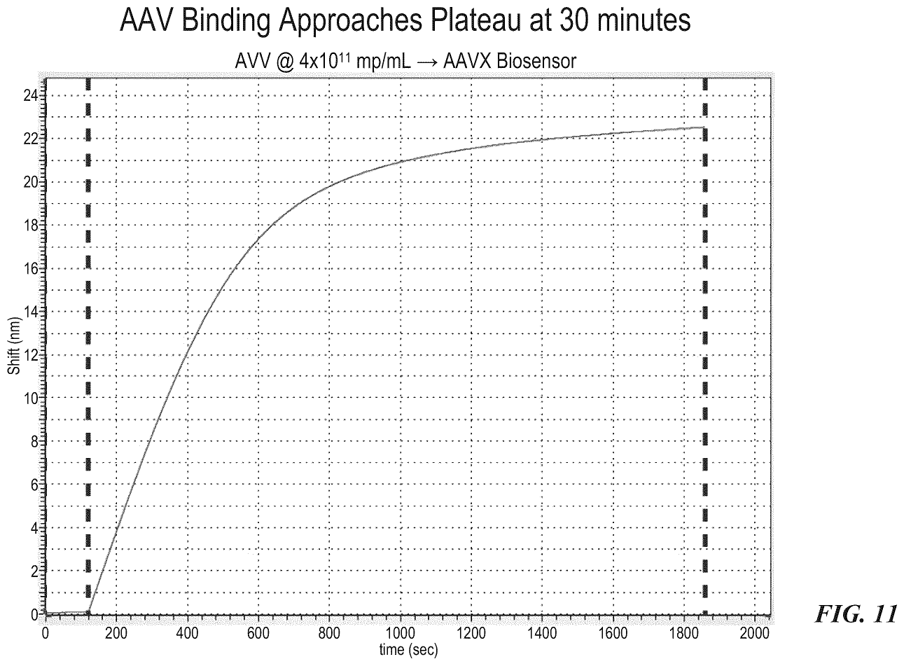

In this example, 200 μl samples of mixtures of empty and full AAV capsids bound with AAVX biosensors for ½ hour at 1000 orbital rpm in a Gator Prime Instrument. FIG. 11 shows typical nm shifts for capture of AAV at 4×10¹¹ viral particles/ml, which approached plateau binding on a AAVX sensor after 30 minutes as monitored by GatorPrime.

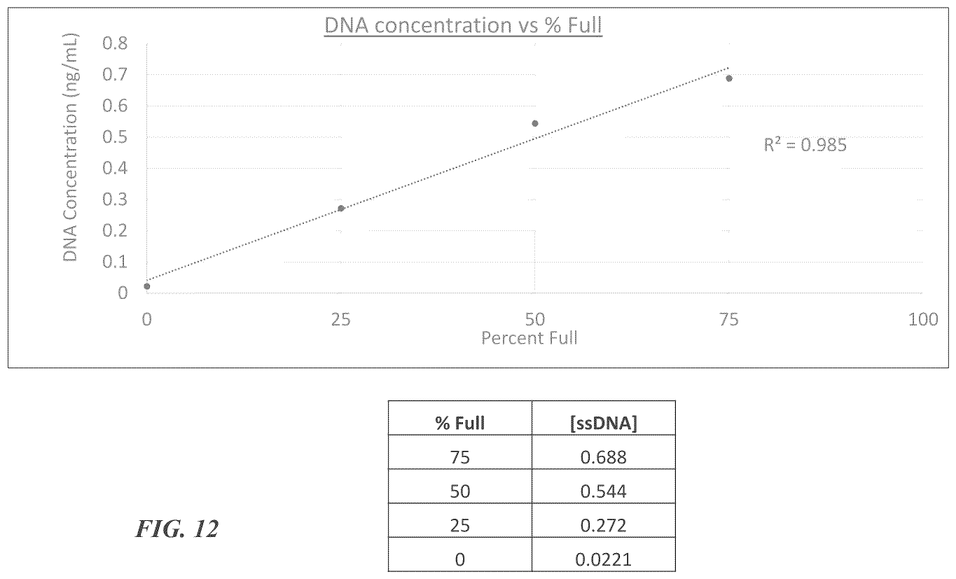

Mixtures of empty and full capsids at 0%, 25%, 50% and 75% full in PBS were captured by AAVX and produced respectively similar and consistent nm shifts of 20.0, 21.8, 22.2, and 22.5. This shows that the capture of the different AAV samples was consistent in the first step. The consistent nm shift indicates that comparable amounts of capsids were captured by the probes in the AAV samples. After a wash step in 200 μL PBS, the probes were then immersed in 200 μL of lysis reagent (PBS, pH 7.4, 3% Tween 20) and placed in a laboratory heating block and subjected to 90° C. for 5 min and followed by a cool down to room temperature for 2 minutes. The AAVX probes were removed and the samples containing the capsid DNA were placed back in the Gator® Prime instrument and assays for ssDNA as described in Example 7 were performed. FIG. 12 depicts results with the DNA levels for the various % full AAV capsids. The DNA levels correlated to the % full capsids, R² was 0.985.

### Example 10: Preparation of SSB Probes

Quartz probes, 1 mm diameter and 2 cm in length, with BLI optical layer at the distal tip were coated with aminopropylsilane using a chemical vapor deposition process (Yield Engineering Systems, 1224P) following manufacturer's protocol. FIGS. 1B and 1C show the BLI probe. The probe tip was immersed in a solution of murine monoclonal anti-fluorescein-crosslinked FICOLL® (from Example 5) at 10 μg/ml in PBS at pH 7.4. After allowing the antibody to adsorb to the probe for 15 minutes, the probe tip was washed in PBS.

The probe tip was then immersed in a solution containing fluorescein-labeled SSB (ThermoFisher: 70032Z500UG). The SSB was fluorescein labeled by a standard method. The probe tip was immersed in a solution a solution of fluorescein-labeled SSB at 10 μg/ml. After 10 minutes, the probe was washed with PBS, then dried at 37 C for 20 min.

### Example 11: DNA Assay with SSB-coated Probes (BLI)

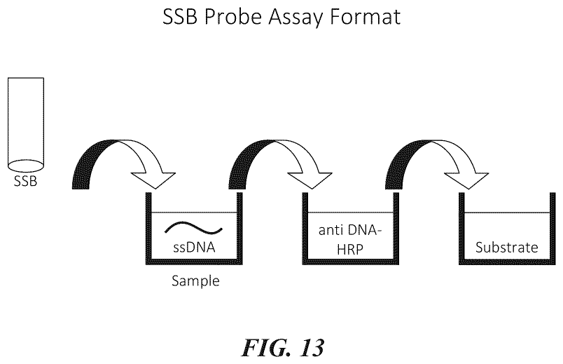

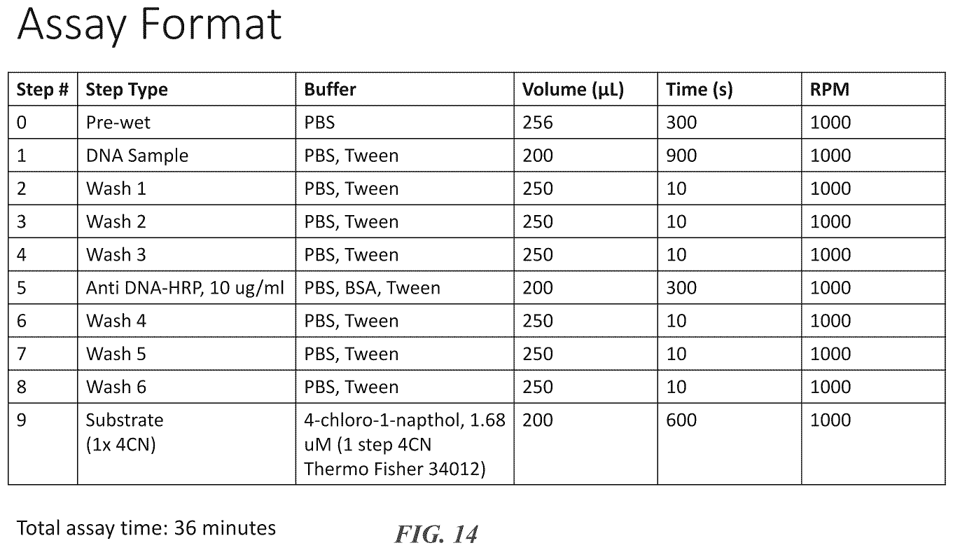

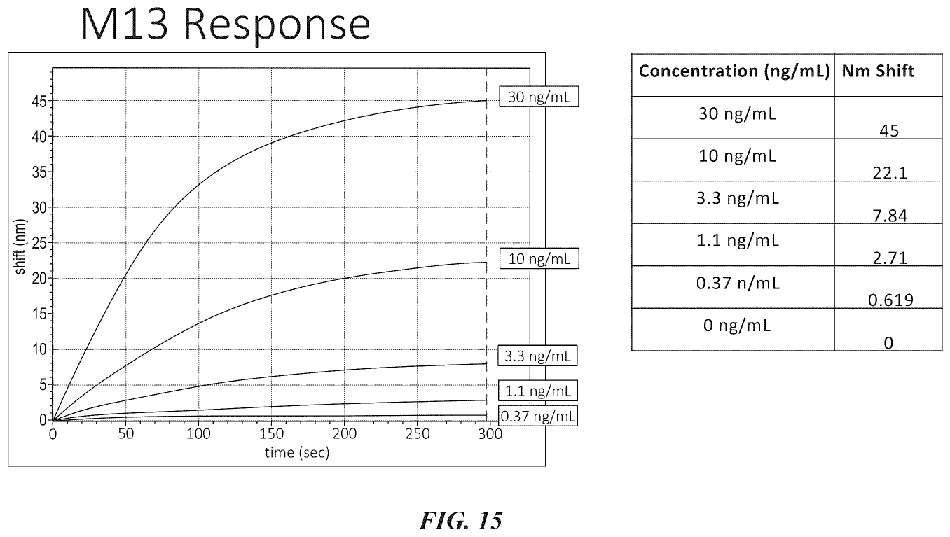

FIG. 13 shows the SSB probe assay format. FIG. 14 tabulates the assay protocol and FIG. 15 presents the M13 plasmid DNA dose response.

### Example 12: Normalization Method (BLI)

In the following example, we illustrate how we use the method of normalization to calculate the empty/full ratio of a AAV sample diluted to different concentrations.

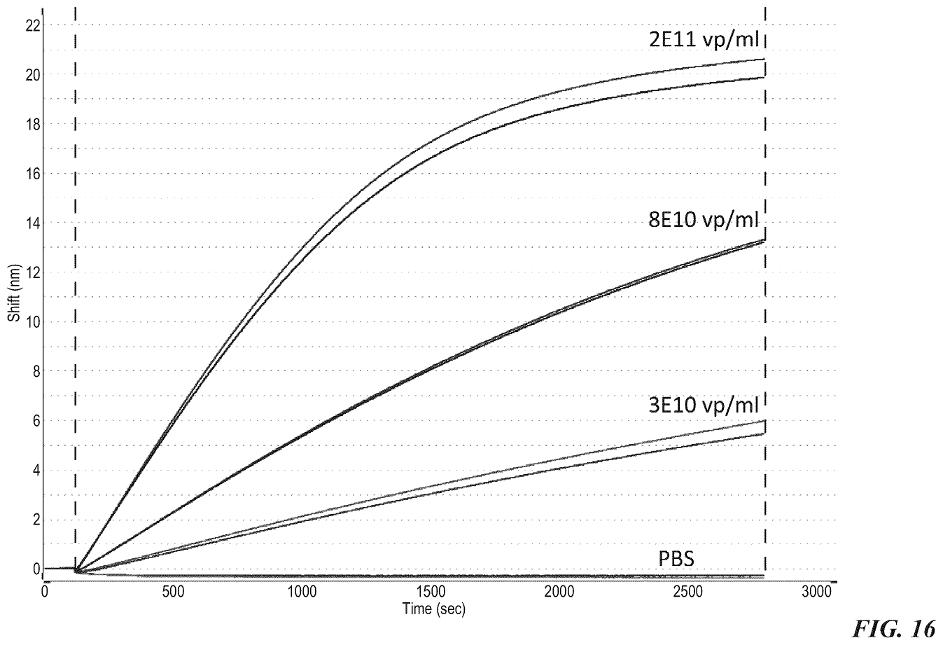

AAV8 sample (˜50% full ratio) was diluted by PBS to generate three concentrations at 2E11, 8E10 and 3E11 vp/ml. Since only PBS was used for dilution, these samples contained the same empty/full ratio but different concentrations. We used the AAVX probes to capture capsids at these different AAV concentrations in duplicate. PBS was used as a control. The corresponding binding curves and data are shown in below. Since the BLI capture signal increases with the AAV concentration, the binding data in FIG. 16 show the following order of wavelength shift 2E11>8E10>3E10 vp/ml as expected.

By using software, we extracted the end point of each binding curve in FIG. 16 and averaged the duplicate BLI data as shown in Table 3. To yield the corresponding normalization factor (last column), the highest average BLI data point (i.e. 20.30) was selected and divided by the average BLI data at each concentration. As a result, the samples at 2E11, 8E10 and 3E10 vp/ml yielded the normalization factors of 1.00, 1.53 and 3.52 respectively.

TABLE 3
AAV Capture Wavelength Shift

| | Conc | BLI | BLI | Ave | Normalization |
|---|---|---|---|---|---|
| Sample | (vp/ml) | #1 | #2 | BLI | Factor |
| AAV8 | 2.00E+11 | 20.68 | 19.91 | 20.30 | 1.00 |
| | 8.00E+10 | 13.36 | 13.24 | 13.30 | 1.53 |
| | 3.00E+10 | 6.01 | 5.52 | 5.77 | 3.52 |
| | PBS | 0 | 0 | 0 | 0 |

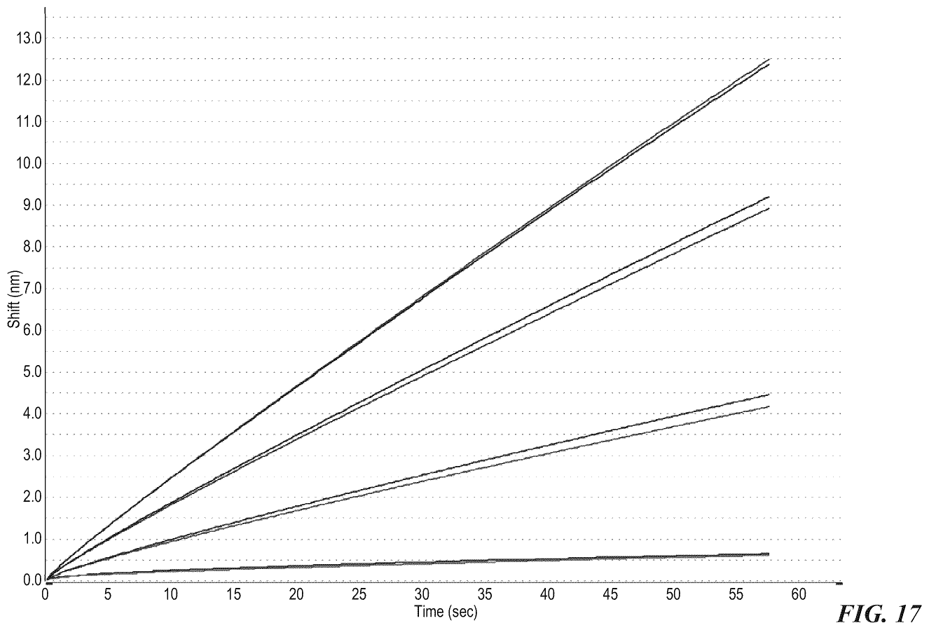

The captured AAV capsids at 2E11, 8E10 and 3E11 vp/ml were lysed and produced the following signal curves in the ssDNA assay according to Example 7A, except the wavelength shifts were read at 60 seconds. The results are shown in FIG. 17.

By using Gator software, we extracted the data point of each signal curve in FIG. 16 and averaged the duplicate BLI data. The average BLI data is then subtracted by the PBS control to yield the net BLI signal. The Net BLI signal is multiplied by the normalization factor from Table 3 to yield the normalized shift values in Table 4.

TABLE 4
ssDNA Capture Wavelength Shift

| | Conc | Ave | Net | Normalization | Normalized |
|---|---|---|---|---|---|
| Sample | (vp/ml) | BLI | BLI | Factor | Shift |
| AAV8 | 2.00E+11 | 11.60 | 11.10 | 1.00 | 11.10 |
| | 8.00E+10 | 8.42 | 7.92 | 1.53 | 12.11 |
| | 3.00E+10 | 3.96 | 3.46 | 3.52 | 12.18 |
| | PBS | 0.50 | 0 | 0 | 0 |

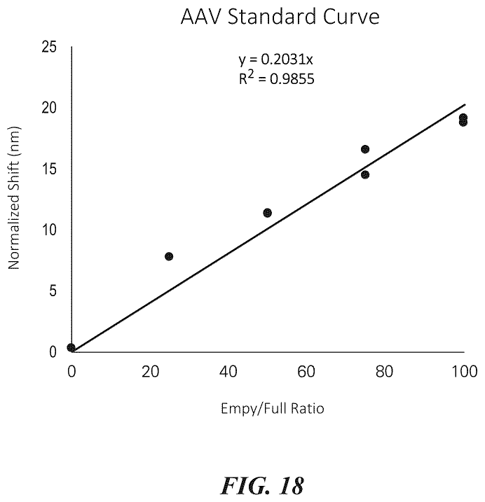

Then the normalized shift values were used to determine the empty/full ratio of the samples at different concentrations. From the AAV standard curve prepared by known standards (FIG. 18), the determined empty/full ratios of the samples at 2E11, 8E10 and 3E10 vg/ml are 54.7%, 59.6 and 59.9% respectively. Overall, the mean empty/full ratio is 58.1% and CV is only 5.0%.

In the present example, we prepared samples containing the same empty/full ratio of AAV capsids but different concentrations and tested to see if normalization of the BLI data could be reliably used to determine the empty/full ratio of the samples. In summary, the determined empty/full ratio of each sample at different AAV concentrations is very similar to each other (5.0% of CV) by using the normalization factor. The result indicates that the normalization procedure can be used to compensate the sample AAV concentration difference and reliably calculate the empty/full ratio of the AAV capsids.

### Example 13. Preparing Anti-DNA-HRP Conjugate

Anti-ssDNA/dsDNA IgG (Absolute Antibody, M3D8, mouse IgG, Cat #Ab00347-1.1) (1 mg in 1 mL PBS) was reacted with SMCC (molar coupling ratio MCR=8, 20 mg/mL in DMF, 0.89 μL, Thermo Scientific) for 1 hour and then purified with Zeba™ Spin Desalting Columns (7k MWCO, Thermo Scientific). Horseradish peroxidase (HRP, Thermo Scientific, MCR=6 to anti-DNA) (1.6 mg in 80 μL PBS) was reacted with Traut's reagent (MCR=8, 20 mg/mL in water, 1.65 μL, Thermo Scientific) for 1 hour and then purified with Zeba™ Spin Desalting Columns. The resulting anti-DNA-SMCC and HRP-thiol solutions were mixed, reacted for 3 hours, and then quenched with 12.5 μL of N-ethyl maleimide (NEM, Thermo Scientific, Cat #23030) in water (20 mg/mL) for 0.5 hour. After passing the conjugate with Zeba™ Spin Desalting Columns, anti-DNA-HRP in PBS was obtained for ssDNA detection solution.

### Example 14: Preparing Streptavidin-Coated Magnetic Particles for AAV Capture

Magnetic particles were used as a platform for streptavidin conjugation. To achieve this, 1 mg of acid-coated magnetic particles (Ocean NanoTech, diameter 1 m) were suspended in 1 mL of 50 mM MES buffer (pH=6) containing 4 mM 1-(3-dimethylaminopropyl)-3-ethylcarbodiimide hydrochloride (EDC) and 10 mM N-hydroxysuccinimide (NHS) and allowed to react for 30 minutes. The magnetic particles were then isolated using a magnet and the supernatant was discarded. Next, 50 g of streptavidin (SA, Agilent) in PBS buffer (pH=7.4, 1 mL) was added to the magnetic particles and allowed to react for 2 hours. The magnetic particles were again isolated using a magnet and the supernatant was discarded. To quench the excess NHS esters, 50 mM Tris buffer (pH=8, 1 mL) was added to the magnetic particles and allowed to react for 30 minutes. Finally, the magnetic particles were isolated using a magnet and the supernatant was discarded. The suspension of magnetic particles conjugated with 50 g/mL SA were obtained by adding 1 mL of PBS buffer.

### Example 15. Preparing Anti-AAV Magnetic Particles

A 7 μL aliquot of Biotin-anti-AAVX (Thermo, Capture Select) at 0.1 mg/ml was added to 100 μL of streptavidin-coated magnetic particles (Example 14) at 1 mg/ml, and incubated at room temperature for 30 minutes with orbital mixing. Particles were then washed by pelleting with a magnet, supernatant removed, then resuspended with 200 μL of PBS. Three wash cycles were performed.

### Example 16. Measuring DNA Content in AAV Samples (BLI)

### Magnetic Particle Capture

Two AAV samples (Virovek, full #AAV8-CMV-GFP) were prepared by initially titering with GatorBio AAV Probes (#160017) following manufacturer's protocol and diluting to 1.4×10¹¹ vp/ml and 6.1×10⁹ vp/ml in PBS buffer. 25 μL aliquots of the anti-AAV-coated magnetic particles (Example 15) were added to the 500 μL AAV samples and incubated at room temperature (RT) for 30 minutes. The particles were then pelleted with a magnetic. 100 μL of the supernatant was removed and assayed with GatorBio AAV Probes to determine the uncaptured capsids in the supernatant. Results from the starting capsid concentration before and after magnetic particle capture enabled a calculation of the captured capsids, which were respectively, 3.7×10¹⁰ vp and 3.1×10⁹ vp. The remaining supernatant was then removed from the samples.

### Lysis

100 μL of a lysis reagent (PBS+3% Tween 20) was added to the magnetic particles and heated at 95° C. for 5 minutes.

ssDNA Assay

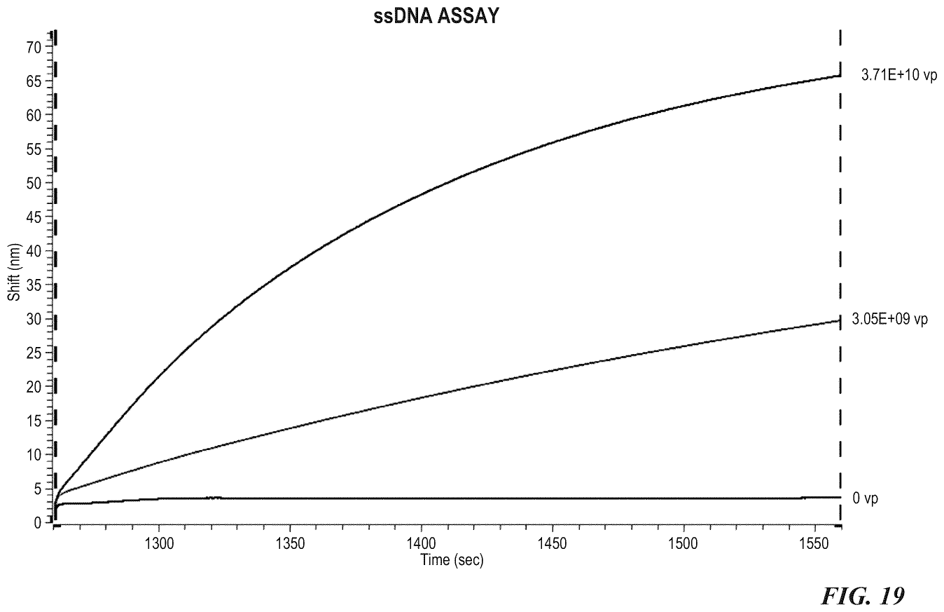

After lysis, the 100 μL lysis reagent containing lysed AAV were transferred to a microplate and ssDNA measured with the ssDNA assay in the GatorBio AAV Ratio kit (#35004) following the manufacturer's protocol using the Gatro Plus BLI reader. Results are shown in FIG. 19.

This example illustrates that that the combination of magnetic particle capture, lysis and ssDNA detection with a BLI biosensor can be used to measure DNA in AAV samples.

### Example 17. Preparation of Biotinylated-Anti-DNA Antibody

Recombinant monoclonal antibody to ssDNA/dsDNA DNA was obtained from Absolute Antibody. Anti-DNA antibody was reacted with EZ-link NHS-PEG4-biotin (Thermofisher scientific) in a biotin to antibody molar ratio of 2 to 1 for 30 minutes at room temperature and then purified by a Zebra Spin Desalting Column (molecular weight cut-off 7K Daltons, Thermofisher Scientific) to obtain biotinylated-anti-DNA antibody (average about 2 biotins/antibody).

### Example 18: DNA Assay with SSB-Coated Probe by Fluorescent Detection

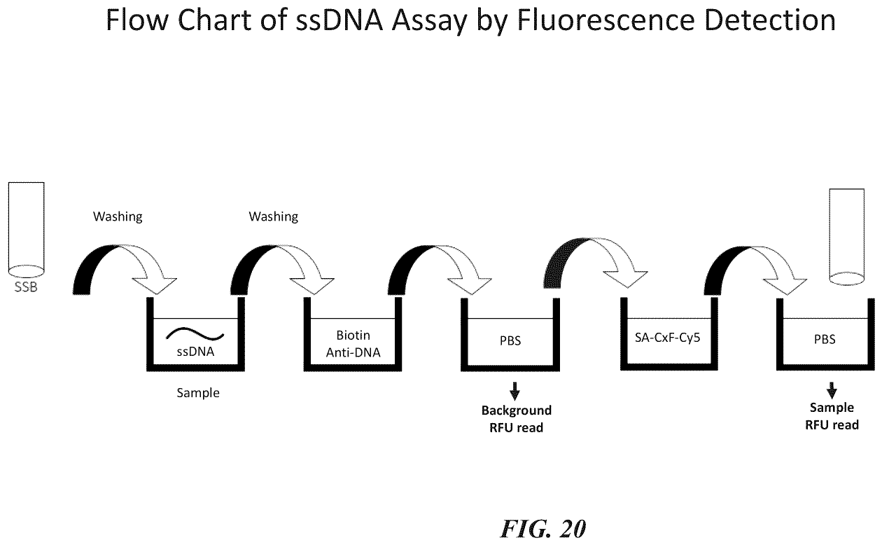

FIG. 20 illustrates a flow chart in performing an assay for ssDNA by fluorescent detection. The SSB-coated probe (Example 10) was immersed in a ssDNA sample, followed by a wash, then immersed in biotin-anti-DNA antibody, followed by a wash, then immersed in a streptavidin-crosslinked FICOLL®-Cy5 conjugate solution. The fluorescent conjugate binds to the probe, and then the probe is moved to a vessel containing a solution for fluorescent detection.

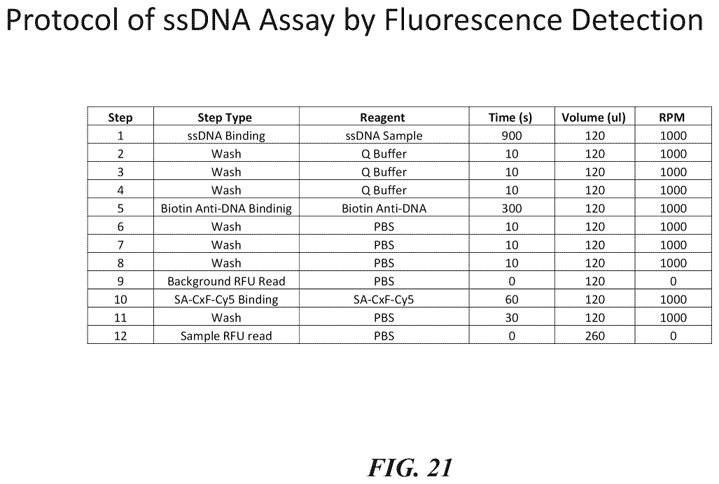

FIG. 21 shows the details in performing a ssDNA assay by fluorescent detection, i.e. reagent concentrations, volumes, time, etc.

### Example 19: DNA Assay of an AAV Sample with SSB-Coated Probe by Fluorescent Detection

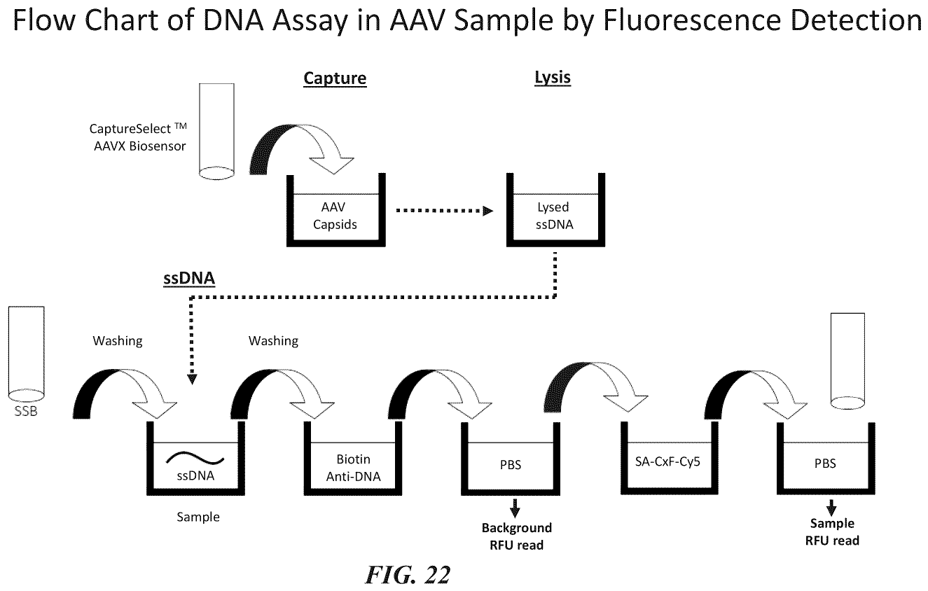

FIG. 22 illustrates a flow chart in performing an assay for ssDNA from a sample containing AAVs by fluorescent detection. FIG. 22 outlines the 3 major steps: AAV capture, lysis, then total AAV DNA measurement by fluorescent detection. The AAV capture step employs an AAVX biosensor to specifically capture AAV in the sample. The AAVX has the probe tip coated with a camelid nanobody (CapSelect™, ThermoFisher) that binds AAV. The capture is allowed to proceed until the maximum binding capacity of the probe is met. After capsid capture and a wash sequence the probe is transferred to a microwell containing a lysis reagent to release the DNA from the capsid. After the lysis step the AAVX probe is removed and an assay for ssDNA is performed as described in Example 18. Since the same number of capsids were initially captured, the ssDNA concentration in the lysed samples is proportional to the E/F ratio.

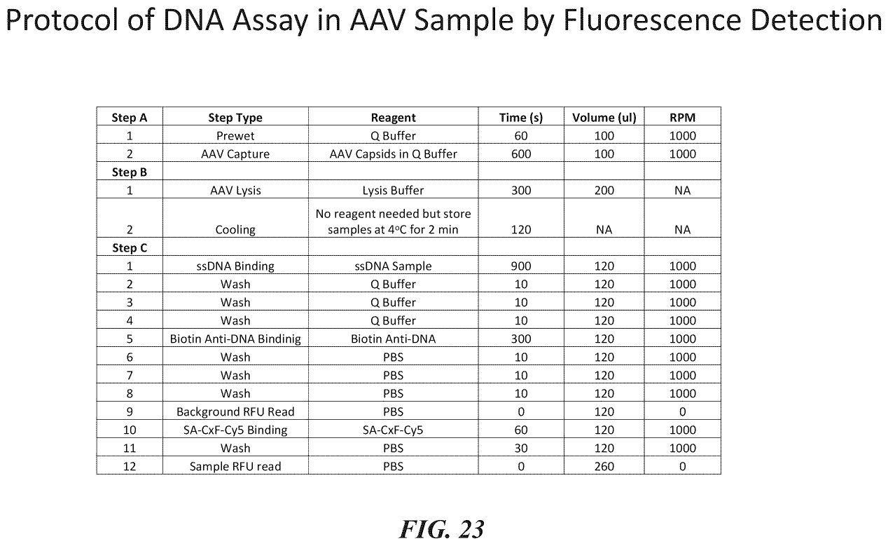

FIG. 23 shows the details in performing a ssDNA assay in an AAV sample by fluorescent detection, i.e. reagent concentrations, volumes, time, etc.

### Example 20. Results of AAV Samples by Fluorescent Detection

AAV samples were prepared by diluting an AAV sample at 1E11 vp/ml to different ratios (5, 25, 50, 75, and 100%) in PBS buffer containing BSA and Tween 20. The samples were lysed and analyzed according to the protocols of FIG. 23. The results are shown in Table 5 and plotted as shown in FIG. 24.

TABLE 5

| AAV Sample | | | Net | Average Net |
|---|---|---|---|---|
| Ratio (%) | Background | RFU | RFU | RFU |
| 0 | 545 | 649 | 104 | 106 |
| 0 | 566 | 674 | 108 | |
| 25 | 579 | 895 | 316 | 329 |
| 25 | 562 | 904 | 342 | |
| 50 | 533 | 1019 | 486 | 497.5 |
| 50 | 542 | 1051 | 509 | |
| 75 | 604 | 1366 | 762 | 687.5 |
| 75 | 545 | 1158 | 613 | |
| 100 | 518 | 1269 | 751 | 778.5 |
| 100 | 562 | 1368 | 806 | |

FIG. 24 shows a linear standard carve, which indicates that the fluorescence signal increased proportionally with the ratios of the full AAV samples.

### Example 21: Preparation of Magnetic Particles Coated with Biotin-Anti-AAVX (Prophetic Example)

Magnetic beads from different vendors with streptavidin surface coating are used. Amount of magnetic beads usage are estimated from the binding capacity reported from the vendors. For Dynabeads MyOne Magnetic beads with Streptavidin coating, the particles contain 10 mg/mL (˜7-10×10⁹ beads/ml) in phosphate buffer saline (PBS), pH 7.4 with 0.01% Tween 20, 0.09% sodium azide as preservative. The beads are resuspended in the vial with 10-20 sec vortex. 10 μL of the particles are pipetted to the 96 well plate or tubes. Equal volume of washing buffer (PBS) is mixed with the beads and using magnetic separation to remove the supernatant. Repeat the washing step for 3 times. Typically, 0.1 mg of the Dynabeads (10 μL) is able to bind 2 μg of biotinylated proteins. Estimated amount of Biotin Anti-AAVx Conjugate (CaptureSelect™) is added to the suspended streptavidin surface functionalized magnetic particles and incubated for 15 min at room temperature with gentle mixing. The mixture is placed to the magnetic separation device for removal of supernatant. The coated beads are washed with a washing buffer for 3-4 times and resuspended to the 200 μL of PBS for AAV capsid capture experiments.

### Example 22. Preparation of Magnetic Particles Coated with Biotin-Single Strand DNA Binding Protein (Prophetic Example)

Similar to Example 13, Dynabeads MyOne Magnetic beads with streptavidin coating are washed and prepared as aforementioned procedure. Washed beads are treated with biotinylated-SSB with estimation of 0.1 mg of the Dynabeads (10 μL) bound to 2 μg of Biotinylated proteins. Incubation of the mixture for 15 min at room temperature with gentle mixing. At the end, the supernatant is removed by magnetic separation and the coated beads are washed with washing buffer for 3-4 times and resuspended to the 200 μL PBS for ssDNA binding assay.

### Example 23. Detection of ssDNA with Magnetic Particles (Prophetic Example)

### AAV Capture Procedure:

Calculate the volume of the empty and full AAV capsids required to prepare 200 μL of 2E11 vp/ml at 0, 20, 40, 60, 80 and 100% full capsid ratios. Capsids can be diluted using PBS.
Adjust the test AAV sample (unknown E/F ratio) to 2E11 vp/ml and use 200 μL for assay.
Optional: As a positive control, prepare 200 μL of 10 ng/ml of M13 ssDNA in PBS and PBS only is used as a reference control.
Transfer 200 μL of each sample into an Eppendorff tube separately.
Add 10 μL of the CaptureSelect™ magnetic particles into each Eppendorff tube.
Vortex the tube briefly and incubate the sample for 30 min at room temperature.
After 30 min, put the tubes in a magnetic rack and wait for 30 sec.
Pipette to remove the sample solution as much as possible by using magnetic separation.
Add 500 ul of PBS into the tube and repeat the washing step for 3 times.
After washing, add 200 μL of lysis buffer (PBS with 3% Tween-20) into each tube.

### AAV Lysis Procedure:

Put the tubes into a heat block already set to 70° C. and wait for 5 min to release ssDNA.
After 5 min, put the tubes aside and allow them to cool down at room temperature.
Put the Eppendorff tubes in a magnetic rack and wait for 30 sec.
Pipette to remove the solution from the beads by magnetic separation and transfer the solution into a new Eppendorff tube separately.
   ssDNA Detection Procedure:
Add 10 μL of magnetic particles coated with SSB into each tube.
Vortex the tube briefly and incubate the sample for 30 min at room temperature.
After 30 min, put the tubes in a magnetic rack and wait for 30 sec.
Pipette to remove the sample solution as much as possible by using magnetic separation.
Add 500 μL of PBS into the tube and repeat the washing step for 3 times.
After washing, add 200 μL of PBS into the tube.
Add 10 μl of anti-ssDNA/dsDNA-HRP conjugate into each tube.
Vortex briefly and incubate at room temperature for 15 min.
After 15 min, put the tubes in a magnetic rack for 30 sec.
Pipette to remove the solution by using magnetic separation and keep the beads.
Add 500 ul of PBS into the tube containing the beads and repeat the washing step for 3 times.
After washing, add 100 μL of PBS into the tube.
Vortex briefly and transfer the sample into the desired wells of a 96-well flat bottom polystyrene microplate.
Add 100 μL of the TMB substrate into each well.
Tap or shake the plate to ensure complete mixing.
Add 50 μL of a stop solution to stop the reaction after 15 min.
Measure the O.D. at 450 nm.
Plot the O.D. reading at 450 nm vs. capsid % to generate the standard curve. The O.D. reading at 450 nm of the test sample can be used to determine the capsid % from the standard curve.

### Example 24. Preparation of Anti-ssDNA/dsDNA Antibody Conjugated to FITC (Prophetic Example)

Anti-ssDNA/ds DNA antibody is conjugated to FITC and purified using the EZLabel™ protein FITC labeling kit from BioVision.
Reconstitute one vial of EZLabel™ FITC with 5-10 μL of DMSO.
Add reconstituted EZLabel™ FITC solution into an Eppendorff tube containing 100 μL (up to 1 mg) of the anti-ssDNA IgG Ab and vortex briefly.
Incubate at room temperature (RT) on rotary shaker for 1 hr.
After incubation, add 20 μL EZLabel™ Quenching Buffer to quench the reaction
Incubate again at RT for 30 min.
Prepare and wash the EZLabel™ Spin Column according to the manufacturer's instruction.
Load the FITC labeled mixture to the EZLabel™ Spin Column.
Spin the column and elute the purified FITC-labeled anti-ssDNA/dsDNA antibody.

### Example 25. ssDNA Detection by Magnetic Particles and Fluorescent Labels (Prophetic Example)

Add 10 μL of magnetic particles coated with SSB into each tube.
Vortex the tube briefly and incubate the sample for 30 min at room temperature.
After 30 min, put the tubes in a magnetic rack and wait for 30 sec.
Pipette to remove the sample solution as much as possible by using magnetic separation.
Add 500 μL of PBS into the tube and repeat the washing step for 3 times.
After washing, add 200 ul of PBS into the tube.
Add 10 μL of the FITC-labeled anti-ssDNA/dsDNA antibody into each tube.
Vortex briefly and incubate at room temperature for 15 min.
After 15 min, put the tubes in a magnetic rack for 30 sec.
Pipette to remove the solution by using magnetic separation and keep the beads.
Add 500 μL of PBS into the tube containing the beads and repeat the washing step for 3 times.
After washing, add 100 μL of PBS into the tube.
Vortex briefly and transfer the sample into the desired wells of a white 96-well flat bottom white opaque microplate.
Tap or shake the plate to ensure complete mixing.
Measure the fluorescence of the wells with an excitation and emission wavelengths at 494 nm and 520 nm respectively.
Plot the fluorescence reading vs. capsid % to generate the standard curve. The fluorescence reading of the test sample can be used to determine the capsid % from the standard curve.

The invention, and the manner and process of making and using it, are now described in such full, clear, concise and exact terms as to enable any person skilled in the art to which it pertains, to make and use the same. The foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed is:

1. A method of determining DNA concentration in DNA viruses, comprising the steps of:
(a) obtaining a first probe having anti-virus antibody immobilized on the tip of the probe, wherein the virus is adeno-associated virus (AAV), adenovirus, or herpes simplex virus type 1 (HSV-1);
(b) dipping the first probe tip in a sample solution comprising a virus sample to capture the virus on the probe in a defined binding condition;
(c) dipping the first probe in a wash solution to wash the first probe tip;
(d) dipping the first probe in a lysis solution and lysing the virus from the first probe tip to obtain single-stranded DNA (ssDNA), and then removing the first probe from the lysis solution;
(e) dipping a second probe comprising a first protein on the probe tip in the lysis solution, wherein the first protein binds to ssDNA;
(f) dipping the second probe in a reagent solution comprising HRP-labelled second protein, wherein the second protein binds to ssDNA;
(g) dipping the second probe in a HRP substrate solution to bind the substrate to the HRP bound on the second probe for a period of time; and
(h) determining the ssDNA concentration in the sample by measuring the wavelength shift due to light interference, and quantitating the wavelength shift against a calibration curve to determine the ssDNA concentration.

2. The method of claim 1, wherein the virus is AAV.

3. The method of claim 1, where in step (b), the virus is captured until the maximum binding capacity of the anti-virus antibody on the probe is met.

4. The method of claim 1, where the first protein and the second protein are independently an antibody against ssDNA or single-stranded DNA binding protein (SSB).

5. The method of claim 1, where the first protein and the second protein are independently an antibody against ssDNA or single-stranded DNA binding protein (SSB).

6. The method of claim 1, wherein the diameter of the tip surface of the second probe is ≤5 mm.

7. A method of determining DNA concentration in DNA viruses, comprising the steps of:
(a) obtaining a first probe having a fixed amount of anti-virus antibody immobilized on the tip of the probe, wherein the virus is AAV, adenovirus, or HSV-1;
(b) dipping the first probe tip in a sample solution comprising a virus sample to capture the virus on the probe;
(c) dipping the first probe in a wash solution to wash the first probe tip;
(d) dipping the first probe in a lysis solution and lysing the virus from the first probe tip to obtain single-stranded DNA (ssDNA), and then removing the first probe from the lysis solution;
(e) dipping a second probe comprising a first protein on the probe tip in the lysis solution, wherein the first protein binds to ssDNA;
(f) dipping the second probe in a reagent solution comprising biotin-labelled second protein, wherein the second protein binds to ssDNA;
(g) dipping the second probe in a conjugate solution comprising a conjugate comprising streptavidin and horse radish peroxidase (HRP), to bind the conjugate to the second probe,
(h) dipping the second probe in a HRP substrate solution to bind the substrate to the HRP in the conjugate for a period of time; and
(i) determining the ssDNA concentration in the sample by measuring the wavelength shift due to light interference, and quantitating the wavelength shift against a calibration curve to determine the ssDNA concentration.

8. The method of claim 7, where the first protein and the second protein are independently an antibody against ssDNA or single-stranded DNA binding protein (SSB).

9. The method of claim 7, wherein the conjugate in step (g) comprises (i) streptavidin, HRP and a polymer, or (ii) streptavidin and crosslinked HRP, or (iii) crosslinked streptavidin and HRP.

10. The method of claim 9, wherein the conjugate comprises streptavidin, HRP and a polymer, and the polymer has a molecular weight over 1 million.

11. The method of claim 10, wherein the polymer is crosslinked copolymers of sucrose and epichlorohydrin.

12. The method of claim 7, wherein the diameter of the tip surface of the second probe is ≤5 mm.

13. A method of measuring the percentage of full virus capsids in a sample, comprising the steps of:
(a) obtaining a first probe having a fixed amount of anti-DNA virus antibody immobilized on the tip of the probe, wherein the virus is adeno-associated virus (AAV), adenovirus, or herpes simplex virus type 1 (HSV-1);
(b) dipping the first probe tip in a sample solution comprising a DNA virus sample to capture the virus capsid on the probe in a defined binding condition for a period of time to measure a first wavelength shift due to light interference as a result of the binding virus on the probe;
(c) determining a normalization factor based on the first wavelength shift of the sample in comparison with (i) the first wavelength shift of a calibrator, or (ii) the average of the first wavelength shifts of all calibrators;
(d) dipping the first probe in a wash solution to wash the first probe tip;
(e) dipping the first probe in a lysis solution and lysing the AAV from the first probe tip to obtain single-stranded DNA (ssDNA), and then removing the first probe from the lysis solution;
(f) dipping a second probe comprising a first protein on the probe tip in the lysis solution, wherein the first protein binds to ssDNA;
(g) dipping the second probe in a reagent solution comprising HRP-labelled second protein, wherein the second protein binds to ssDNA;
(h) dipping the second probe in a HRP substrate solution to bind the substrate to the HRP bound on the second probe for a period of time and measuring the second wavelength shift due to light interference;
(i) applying the normalization factor to the second wavelength shift to produce a normalized second wavelength shift, and
(j) quantitating the normalized second wavelength shift against a calibration curve having normalized wavelength shift plotted against % of full AAV capsids to determine the percentage of full AAV capsids in the sample.

14. The method of claim 13, wherein the normalization factor in step (c) is determined by comparing the first wavelength shift of the sample in comparison with the average of the first wavelength shifts of all calibrators.

15. The method of claim 13, wherein the calibration curve is established by measuring the normalized shifts of calibrator samples with known percentages of full AAV capsids and plotting normalized wavelength shifts against percentages of full AAV capsids.

16. A method of determining DNA concentration in DNA viruses, comprising the steps of:
(a) obtaining a first solid surface immobilized with a fixed amount of anti-virus antibody, wherein the virus is adeno-associated virus (AAV), adenovirus, or herpes simplex virus type 1 (HSV-1);
(b) contacting the first solid surface with a sample solution comprising a virus sample to capture the virus on the first solid surface in a defined binding condition;
(c) washing the first solid surface with a wash solution to remove the non-bound materials from the solid surface;
(d) contacting the first solid surface with a lysis solution and lysing the virus from the first solid surface to obtain single-stranded DNA (ssDNA) in the lysis solution, and then collecting the lysis solution;
(e) contacting the ssDNA-containing lysis solution with a second solid surface immobilized with a first protein, wherein the first protein binds to ssDNA;
(f) washing the second solid surface;
(g) contacting the second solid surface with a second protein conjugated with a fluorescent label; and
(h) detecting the fluorescent label and determining the DNA concentration in the virus; wherein the first protein and the second protein are independently SSB or anti-DNA antibody.

17. The method of claim 16, wherein step (g) comprises:
(g1): contacting the second solid surface with the second protein conjugated with a biotin, and then
(g2) contacting the second solid surface with a conjugate comprising streptavidin and the fluorescent label for detection.

18. The method of claim 17, wherein the conjugate in step (g2) comprises streptavidins, fluorescent molecules, and copolymers of sucrose and epichlorohydrin.

19. The method of claim 16, wherein the first protein and the second protein are different.

20. The method of claim 16, wherein the fluorescent label is Cy3, Cy5, or AlexaFluor 647.
